SEQUENCE LISTING (8 sequences)

SEQ ID NO: 1 - DNA, 21 bp, Artificial Sequence
  Primer
aagactgtaaccacgcgtctg

SEQ ID NO: 2 - DNA, 21 bp, Artificial Sequence
  Primer
attccatacctgttcaccgac

SEQ ID NO: 3 - DNA, 21 bp, Artificial Sequence
  Primer
atgtcaccacaaacagaggcc

SEQ ID NO: 4 - DNA, 26 bp, Artificial Sequence
  Primer
tcattatttgtagagctcatccatgc

SEQ ID NO: 5 - DNA, 26 bp, Artificial Sequence
  Primer
gcatgattgaacaagatggattgcac

SEQ ID NO: 6 - DNA, 26 bp, Artificial Sequence
  Primer
gaactcgtcaagaaggcgatagaagg

SEQ ID NO: 7 - DNA, 25 bp, Artificial Sequence
  Primer
cgaagtatcgactcaactatcagag

SEQ ID NO: 8 - DNA, 25 bp, Artificial Sequence
  Primer
gactaccttggtgatctcgcctttc

(19) United States
(12) Patent Application Publication
(10) Pub. No.: US 2021/0123067 A1
(43) Pub. Date: Apr. 29, 2021

(54) COMPOSITIONS AND METHODS FOR TRANSFERRING CYTOPLASMIC OR NUCLEAR TRAITS OR COMPONENTS

(71) Applicant: MONSANTO TECHNOLOGY LLC, St. Louis, MO (US)

(72) Inventors: Charles L. Armstrong, O'Fallon, MO (US); Vladimir A. Sidorov, Chesterfield, MO (US)

(21) Appl. No.: 16/497,762

(22) PCT Filed: Apr. 3, 2018
(86) PCT No.: PCT/US2018/025917

§ 371 Date: Sep. 25, 2019

Related U.S. Application Data
(60) Provisional application No. 62/491,913, filed on Apr. 28, 2017.
(60) Provisional application No. 62/480,983, filed on Apr. 3, 2017.

(51) Int. Cl. C12N 15/82; C12Q 1/6895

(52) U.S. Cl. CPC C12N 15/8213; C12Q 2600/158; C12Q 2600/13; C12Q 1/6895

(57) ABSTRACT

The invention provides novel methods and compositions for transfer of nuclear and/or plastomic genomes, or portions thereof, or cytoplasmic component(s) and/or genetic material, between plant cells. Methods for production of a wounded mixed cell culture, or mixing two or more cell cultures after wounding, and transfer of genetic and/or cytoplasmic component(s), such as transfer of nuclear and/or plastid gene(s) or mutations, edits or alleles, between cells of the mixed culture, are also provided. Wounded mixed cell cultures produced by such methods, and resulting cells and regenerated plants, plant parts, and progeny plants are further provided. Molecular and genetic analyses, and screenable and selection markers, are also provided to confirm transfer and presence of cytoplasmic and/or nuclear component(s) and/or gene(s), mutation(s) or allele(s) in cells and plants produced by these methods.

### CROSS-REFERENCE TO RELATED APPLICATIONS

This application claims the benefit of United States Provisional Application Nos. 62/480,983 (filed Apr. 3, 2017), and 62/491,913 (filed Apr. 28, 2017), both of which are herein incorporated by reference in their entirety.

### INCORPORATION OF SEQUENCE LISTING

A computer readable form of a sequence listing is filed with this application by electronic submission and is incorporated into this application by reference in its entirety. The sequence listing is contained in the file named MONS416WO_ST25.txt, which is 1.71 kilobytes in size (measured in operating system MS Windows) and created on Apr. 3, 2018.

### FIELD OF THE INVENTION

The invention relates generally to the fields of agriculture, plant biotechnology, and molecular biology. More specifically, the invention relates to compositions and methods for transferring cytoplasmic, organellar (e.g. plastid-encoded) or nuclear traits between plant cells by cell fusion.

### BACKGROUND

The ability to create plants having novel combinations of genetic traits is useful for improving crop yields and resisting disease and pest pressures. In addition to crossing or breeding plants together, novel combinations of traits can be introduced transgenically or through various mutagenesis techniques. However, many plant species and varieties are difficult to transform, culture and/or regenerate from an explant or plant material. One way of introducing novel traits into a plant that are recalcitrant to transformation or culturing techniques might be to transfer those traits from other germplasms through molecular techniques. Although plastid and nuclear traits have been transferred via protoplast fusion, regeneration of plants from protoplasts remains difficult for many economically important plant species. A need exists in the art for novel and improved methods for transferring genetic and cytoplasmic elements and traits between different plant cells, tissues and varieties to create desired combinations of traits.

### SUMMARY OF THE INVENTION

In one aspect the invention provides a method for transfer of genetic material comprising: a) obtaining a first plant cell culture and a second plant cell culture; b) mixing the first and second plant cell cultures to obtain a mixed cell culture; and c) wounding the cells of the mixed cell culture to produce at least one combined cell into which transfer of a genetic material has occurred following said mixing. In certain embodiments the method further comprises d) screening or selecting for the at least one combined cell, or a progeny cell thereof, or a plant developed or regenerated from the at least one combined cell, or a progeny cell thereof, based on a selectable or screenable marker. In certain embodiments of the method, one or more cells of the first plant cell culture comprise a transgene, native allele, edit or mutation of interest that is not present in the cells of the second plant cell culture.

In some embodiments the at least one combined cell, or a progeny cell thereof, comprises the transgene, native allele, edit or mutation of interest present in the one or more cells of the first plant cell culture. In certain embodiments the first and second plant cell cultures are callus cultures or cell suspension cultures.

The at least one of the first and second plant cell cultures may comprise cells having a plastid genome-encoded marker gene, and/or wherein at least one of the first and second plant cell cultures comprise cells having a nuclear genome-encoded marker gene. In some embodiments the first and second plant cell cultures each comprise cells having a plastid genome-encoded marker gene. The first and second plant cell cultures may also each comprise cells having a nuclear genome-encoded marker gene. In certain embodiments the first plant cell culture comprises cells having a plastid genome-encoded marker gene, and the second plant cell culture comprises cells having a nuclear genome-encoded marker gene.

In some embodiments of the method wherein the at least one of the first and second plant cell cultures may comprise cells having a plastid genome-encoded marker gene, and/or wherein at least one of the first and second plant cell cultures comprise cells having a nuclear genome-encoded marker gene, the method may comprise: d) screening or selecting for the at least one combined cell of the mixed cell culture, or at least one progeny cell thereof, or a plant developed or regenerated from the at least one combined cell, or a progeny cell thereof, based on the presence of the plastid genome-encoded marker gene, during and/or after step (c) or step (d). In some embodiments the method further comprises: d) screening or selecting for the at least one combined cell of the mixed cell culture, or at least one progeny cell thereof, or a plant developed or regenerated from the at least one combined cell, or a progeny cell thereof, based on the presence of the nuclear genome-encoded marker gene, during and/or after step (c) or step (d). In certain embodiments the method further comprises regenerating a plant from the mixed cell culture and/or the at least one combined cell, or at least one progeny cell thereof, before having screened based on the presence of a selectable or screenable marker. The method may also comprise a step of regenerating a plant from the mixed cell culture and/or the at least one combined cell, or at least one progeny cell thereof, after having screened based on the presence of a selectable or screenable marker.

In some embodiments the cells of the first and/or second plant cell cultures are dicot plant cells. In particular embodiments the dicot plant cells are selected from the group consisting of tobacco, tomato, soybean, canola, and cotton cells. In other embodiments the cells of the first and/or second plant cell cultures are monocot plant cells. In particular embodiments the monocot plant cells are selected from the group consisting of corn, rice, wheat, barley, and sorghum cells.

In certain embodiments the plastid genome-encoded marker gene is a selectable marker gene. In particular embodiments the plastid genome-encoded selectable marker gene is selected from the group consisting of: aadA, rrnS, rrnL, nptII, aphA-6, psbA, bar, HPPD, ASA2, and AHAS. In some embodiments the plastid genome-encoded marker gene is a screenable marker gene. In particular embodiments the plastid genome-encoded screenable marker gene is gfp or gus.

In certain embodiments the nuclear genome-encoded marker gene is a selectable marker gene. In particular embodiments the nuclear genome-encoded selectable marker gene is selected from the group consisting of: nptII, EPSPS, bar, hpt, dmo, and GAT. In certain embodiments the nuclear genome-encoded marker gene is a screenable marker gene. In particular embodiments the nuclear genome-encoded screenable marker gene is selected from the group consisting of: uidA (gus) and gfp.

In some embodiments a first cell of the first plant cell culture is a donor cell and a second cell of the second plant cell culture is a recipient cell. In certain embodiments the cells of the first and second plant cell cultures have the same ploidy level. In some embodiments the combined cell and cells of one or both of the first and/or second plant cell cultures have the same ploidy level.

In some embodiments of the method wherein the at least one of the first and second plant cell cultures may comprise cells having a plastid genome-encoded marker gene, and/or wherein at least one of the first and second plant cell cultures comprise cells having a nuclear genome-encoded marker gene, the cells of at least one of the first and second plant cell cultures comprise a plastid genome-encoded marker gene, and wherein cells of at least one of the first and second plant cell cultures comprise a nuclear genome-encoded marker gene.

In certain embodiments of the method wherein the first and second plant cell cultures are callus cultures or cell suspension cultures, the cells of the mixed cell culture, or progeny cells thereof, are screened or selected for the presence of a marker gene encoded by a nuclear genome-encoded gene, during and/or after step (c).

In some embodiments of the methods, the cells of the mixed cell culture, the first plant cell culture and/or the second plant cell culture, or progeny cells thereof, are homoplastomic for a plastid-encoded gene. In certain embodiments of the methods, the cells of the mixed cell culture, the first plant cell culture and/or the second plant cell culture, or progeny cells thereof, are heteroplastomic for a plastid-encoded gene.

In another aspect, the invention provides a combined plant cell produced by a method for transfer of genetic material comprising: a) obtaining a first plant cell culture and a second plant cell culture; b) mixing the first and second plant cell cultures to obtain a mixed cell culture; and c) wounding the cells of the mixed cell culture to produce at least one combined cell into which transfer of a genetic material has occurred following said mixing. In certain embodiments the combined plant cell is a dicot plant cell. In particular embodiments the dicot plant cell is selected from the group consisting of: a tobacco, a tomato, a soybean, a canola, and a cotton plant cell. In other embodiments, the combined plant cell is a monocot plant cell. In particular embodiments the combined plant cell is selected from the group consisting of: a corn, a rice, a wheat, and a sorghum plant cell.

The invention further provides a plant regenerated from the combined plant cell produced by such a method, or a progeny cell thereof. A seed, progeny plant, or progeny seed of the plant is also contemplated.

In certain embodiments the plant is a dicot plant. In particular embodiments the dicot plant is selected from the group consisting of: a tobacco, a tomato, a soybean, a canola, and a cotton plant. In other embodiments the regenerated plant is a monocot plant. In particular embodiments, the monocot plant is selected from the group consisting of: a corn, a rice, a wheat, a barley, and a sorghum plant.

Another aspect of the invention provides a wounded mixed cell culture produced by the method of transfer of genetic material comprising: a) obtaining a first plant cell culture and a second plant cell culture; b) mixing the first and second plant cell cultures to obtain a mixed cell culture; and c) wounding the cells of the mixed cell culture to produce at least one combined cell into which transfer of a genetic material has occurred following said mixing. In certain embodiments the genetic transfer comprises plastid or organellar gene transfer. The genetic transfer may also or alternatively comprise nuclear gene transfer.

Another aspect of the invention provides a method for transfer of genetic material comprising: a) obtaining a first plant cell culture and a second plant cell culture; b) wounding the cells of one or both of the first and second plant cell cultures; and c) mixing the first and second plant cell cultures to obtain a mixed cell culture to produce at least one combined cell into which transfer of a genetic material has occurred. In some embodiments the method further comprises: d) screening or selecting for the at least one combined cell, or a progeny cell thereof, or a plant developed or regenerated from the at least one combined cell, or a progeny cell thereof, based on a selectable or screenable marker. In some embodiments the first and second plant cell cultures are callus cultures or cell suspension cultures. In certain embodiments of such methods, at least one of the first and second plant cell cultures comprises cells having a plastid genome-encoded marker gene, and/or wherein at least one of the first and second plant cell cultures comprise cells having a nuclear genome-encoded marker gene. In some embodiments the first and second plant cell cultures each comprise cells having a plastid genome-encoded marker gene. In certain embodiments the first and second plant cell cultures each comprise cells having a nuclear genome-encoded marker gene. The first plant cell culture may also comprise, in certain embodiments, cells having a plastid genome-encoded marker gene, and wherein the second plant cell culture may comprises cells having a nuclear genome-encoded marker gene.

The method may also further comprise: d) screening or selecting for the at least one combined cell of the mixed cell culture, or at least one progeny cell thereof, or a plant developed or regenerated from the at least one combined cell, or a progeny cell thereof, based on the presence of the plastid genome-encoded marker gene, during and/or after step (c) and/or step (d). The method may also further comprise: d) screening or selecting for the at least one combined cell of the mixed cell culture, or at least one progeny cell thereof, or a plant developed or regenerated from the at least one combined cell, or a progeny cell thereof, based on the presence of the nuclear genome-encoded marker gene, during and/or after step (c) and/or step (d).

The method may further comprise a step of e): regenerating a plant from the mixed cell culture and/or the at least one combined cell, or at least one progeny cell thereof.

In certain embodiments, the cells of the first and/or second plant cell cultures are dicot plant cells. In other embodiments the cells of the first and/or second plant cell cultures are monocot plant cells.

In certain embodiments of such methods, the plastid genome-encoded marker gene is a selectable or screenable marker gene. Further, in some embodiments of the methods, the nuclear genome-encoded marker gene is a selectable or screenable marker gene.

In some embodiments of the method for transfer of genetic material comprising: a) obtaining a first plant cell culture and a second plant cell culture; b) wounding the cells of one or both of the first and second plant cell cultures; and c) mixing the first and second plant cell cultures to obtain a mixed cell culture to produce at least one combined cell into which transfer of a genetic material has occurred, a first cell of the first plant cell culture is a donor cell and a second cell of the second plant cell culture is a recipient cell. In certain embodiments the cells of the first and second plant cell cultures have the same ploidy level. In some embodiments the combined cell and cells of one or both of the first and/or second plant cell cultures have the same ploidy level.

In certain embodiments of such methods, wherein at least one of the first and second plant cell cultures comprises cells having a plastid genome-encoded marker gene, and/or wherein at least one of the first and second plant cell cultures comprise cells having a nuclear genome-encoded marker gene, the cells of at least one of the first and second plant cell cultures comprise a plastid genome-encoded marker gene, and cells of at least one of the first and second plant cell cultures comprise a nuclear genome-encoded marker gene.

The cells of the mixed cell culture, or progeny cells thereof, produced by a method further comprising a step of d): screening or selecting for the at least one combined cell, or a progeny cell thereof, or a plant developed or regenerated from the at least one combined cell, or a progeny cell thereof, based on a selectable or screenable marker, may also screened or selected for the presence of a marker gene encoded by a nuclear genome-encoded gene, during and/or after step (c) or (d).

In another aspect, the invention provides a combined plant cell produced by a method for transfer of genetic material comprising: a) obtaining a first plant cell culture and a second plant cell culture; b) wounding the cells of one or both of the first and second plant cell cultures; and c) mixing the first and second plant cell cultures to obtain a mixed cell culture to produce at least one combined cell into which transfer of a genetic material has occurred. In certain embodiments the combined plant cell is a dicot plant cell. In particular embodiments the dicot plant cell is selected from the group consisting of: a tobacco, a tomato, a soybean, a canola, and a cotton plant cell. In other embodiments the combined plant cell is a monocot plant cell. In particular embodiments the monocot plant is selected from the group consisting of: a corn, a rice, a wheat, and a sorghum plant cell.

A plant regenerated from the combined plant cell produced by such a method, or a progeny cell thereof, is also contemplated, as is a seed, progeny plant, or progeny seed of such a plant.

The invention also provides a wounded mixed cell culture produced by such methods. The genetic transfer may comprise plastid or organellar gene transfer. The genetic transfer may also, or alternatively, comprise nuclear gene transfer.

In another aspect, the invention provides a method for editing a plant cell comprising: a) obtaining a first plant cell culture and a second plant cell culture, wherein one or more cells of the first plant cell culture comprise a recombinant DNA transgene comprising a sequence encoding a site-specific nuclease operably linked to a first promoter; b) mixing the first and second plant cell cultures to obtain a mixed cell culture; and c) wounding the cells of the mixed cell culture to produce at least one edited product cell having an edit or mutation introduced in its genome by the site-specific nuclease. In some embodiments the method further comprises: d) screening or selecting for the at least one edited product cell, or a progeny cell thereof, or a plant developed or regenerated from the at least one edited product cell, or a progeny cell thereof, having the edit or mutation.

In such methods, the plant developed or regenerated from the at least one edited product cell, or a progeny cell thereof, is screened or selected based on a trait or phenotype produced by the edit or mutation and present in the developed or regenerated plant, or a progeny plant, plant part or seed thereof. In particular embodiments the at least one edited product cell, or a progeny cell thereof, or the plant developed or regenerated from the at least one edited product cell, or a progeny cell thereof, is or are screened or selected based on a molecular assay. In some embodiments of the methods, the first and second plant cell cultures are callus cultures or cell suspension cultures. The method may also comprise regenerating a plant from the mixed cell culture and/or the at least one edited product cell, or at least one progeny cell thereof, before or after such screening or selection.

In such contemplated methods, the cells of the first and/or second plant cell cultures may be dicot plant cells. In particular embodiments the dicot plant cells are selected from the group consisting of tobacco, tomato, soybean, canola, and cotton cells. In other embodiments the cells of the first and/or second plant cell cultures are monocot plant cells. In particular embodiments the monocot plant cells are selected from the group consisting of corn, rice, wheat, barley, and sorghum cells.

In certain embodiments, a first cell of the first plant cell culture is a donor cell and a second cell of the second plant cell culture is a recipient cell.

In some embodiments, the first promoter operably linked to the sequence encoding a site-specific nuclease is a constitutive promoter, a tissue-specific or tissue-preferred promoter, a developmental stage promoter, or an inducible promoter. In certain embodiments the site-specific nuclease is a zinc-finger nuclease (ZFN), a meganuclease, an RNA-guided endonuclease, a TALE-endonuclease (TALEN), a recombinase, or a transposase. In particular embodiments the site-specific nuclease is an RNA-guided nuclease.

In an aspect of the present disclosure, methods for editing a plant cell comprise: a) obtaining a first plant cell culture and a second plant cell culture, wherein one or more cells of the first plant cell culture comprise a recombinant DNA transgene comprising a sequence encoding a site-specific nuclease operably linked to a first promoter; b) mixing the first and second plant cell cultures to obtain a mixed cell culture; and c) wounding the cells of the mixed cell culture to produce at least one edited product cell having an edit or mutation introduced in its genome by the site-specific nuclease, the one or more cells of the first plant cell culture further comprise a first recombinant DNA construct comprising a first transcribable DNA sequence encoding a guide RNA molecule operably linked to a promoter. In another aspect, methods for editing a plant cell comprise: a) obtaining a first plant cell culture and a second plant cell culture, wherein one or more cells of the first plant cell culture comprise a recombinant DNA transgene comprising a sequence encoding a site-specific nuclease operably linked to a first promoter; b) wounding the cells of one or both of the first and second plant cell cultures; and c) mixing the first and second plant cell cultures to obtain a mixed cell culture to produce at least one edited product cell having an edit or mutation introduced in its genome by the site-specific nuclease. In certain embodiments of such methods, the one or more cells of the first plant cell culture may further comprise a second recombinant DNA construct comprising a second transcribable DNA sequence encoding a donor template molecule operably linked to a promoter. In particular embodiments the donor template molecule comprises a transgene comprising a coding sequence or transcribable DNA sequence operably linked to a plant-expressible promoter. In certain embodiments the promoter operably linked to the first transcribable DNA sequence is a constitutive promoter, a tissue-specific or tissue-preferred promoter, a developmental stage promoter, or an inducible promoter. In particular embodiments the promoter operably linked to the second transcribable DNA sequence is a constitutive promoter, a tissue-specific or tissue-preferred promoter, a developmental stage promoter, or an inducible promoter.

Further, the one or more cells of the second plant cell culture may comprise a recombinant DNA construct comprising a first transcribable DNA sequence encoding a guide RNA molecule operably linked to a promoter. In some embodiments the one or more cells of the second plant cell culture comprise a recombinant DNA construct comprising a second transcribable DNA sequence encoding a donor template molecule operably linked to a promoter. In certain embodiments the donor template molecule comprises a transgene comprising a coding sequence or transcribable DNA sequence operably linked to a plant-expressible promoter.

Another aspect of the present disclosure provides an edited product cell produced by the method comprising: a) obtaining a first plant cell culture and a second plant cell culture, wherein one or more cells of the first plant cell culture comprise a recombinant DNA transgene comprising a sequence encoding a site-specific nuclease operably linked to a first promoter; b) mixing the first and second plant cell cultures to obtain a mixed cell culture; and c) wounding the cells of the mixed cell culture, thus producing at least one edited product cell having an edit or mutation introduced in its genome by the site-specific nuclease. In another aspect, an edited product cell is provided that is produced by a method comprising: a) obtaining a first plant cell culture and a second plant cell culture, wherein one or more cells of the first plant cell culture comprise a recombinant DNA transgene comprising a sequence encoding a site-specific nuclease operably linked to a first promoter; b) wounding the cells of one or both of the first and second plant cell cultures; and c) mixing the first and second plant cell cultures to obtain a mixed cell culture to produce at least one edited product cell having an edit or mutation introduced in its genome by the site-specific nuclease. In certain embodiments, the edited product cell is a dicot plant cell. In other embodiments, the edited product cell is a monocot plant cell. A plant regenerated or developed from an edited product cell produced by such a method, or a progeny plant cell thereof, is also contemplated. In certain embodiments the regenerated plant is a dicot or monocot plant. A seed, progeny plant, or progeny seed of such a plant is also provided, as is a wounded mixed cell culture produced by such methods.

In another aspect, the invention provides a method for providing a donor DNA sequence to a plant cell comprising: a) obtaining a first plant cell culture and a second plant cell culture, wherein one or more cells of the first plant cell culture comprise a recombinant DNA transgene comprising a sequence encoding a site-specific nuclease operably linked to a first promoter and a donor DNA sequence; b) mixing the first and second plant cell cultures to obtain a mixed cell culture; and c) wounding the cells of the mixed cell culture to produce at least one product cell having an insertion sequence or mutation of the donor DNA sequence introduced in its genome by the site-specific nuclease. In some embodiments the method further comprises: d) screening or selecting for the at least one product cell comprising the insertion sequence or mutation of the donor DNA sequence, or a progeny cell thereof, or a plant developed or regenerated from the at least one edited product cell, or a progeny cell thereof, having the insertion sequence or mutation of the donor DNA sequence. In some embodiments, the donor DNA sequence is a template for templated editing. In other embodiments the donor DNA sequence comprises a transgene.

In an aspect, a plant developed or regenerated from the at least one edited product cell, or a progeny cell thereof, is screened or selected based on a trait or phenotype produced by an insertion sequence or mutation from the donor DNA sequence and present in the developed or regenerated plant, or a progeny plant, plant part or seed thereof. In particular embodiments the at least one product cell comprising the donor DNA sequence, or a progeny cell thereof, or the plant developed or regenerated from the at least one edited product cell, or a progeny cell thereof, is/are screened or selected based on a molecular assay. In some embodiments of the methods, the first and second plant cell cultures are callus cultures or cell suspension cultures. The method may also comprise regenerating a plant from the mixed cell culture and/or the at least one product cell comprising the donor DNA sequence, or at least one progeny cell thereof, before or after such screening or selection.

In such contemplated methods, the cells of the first and/or second plant cell cultures may be dicot plant cells. In particular embodiments the dicot plant cells are selected from the group consisting of tobacco, tomato, soybean, canola, and cotton cells. In other embodiments the cells of the first and/or second plant cell cultures may be monocot plant cells. In particular embodiments the monocot plant cells are selected from the group consisting of corn, rice, wheat, barley, and sorghum cells.

In certain embodiments a first cell of the first plant cell culture is a donor cell and a second cell of the second plant cell culture is a recipient cell.

In some embodiments the first promoter operably linked to the sequence encoding a site-specific nuclease is a constitutive promoter, a tissue-specific or tissue-preferred promoter, a developmental stage promoter, or an inducible promoter. In certain embodiments the site-specific nuclease is a zinc-finger nuclease (ZFN), a meganuclease, an RNA-guided endonuclease, a TALE-endonuclease (TALEN), a recombinase, or a transposase. In particular embodiments, the site-specific nuclease is an RNA-guided nuclease.

### BRIEF DESCRIPTION OF THE DRAWINGS

FIG. 1. Sensitivity of parental lines to antibiotics.

FIG. 2. GFP and GUS expression in leaves and callus of parental lines.

FIG. 3. GFP and GUS expression in the selected cell line #IV and expression of GUS in the selected cell line #III.

FIG. 4. Comparison of whole plant and flower morphology of a regenerated tobacco plant from the #IV cell line in comparison to the 30125 and 42061 parental lines.

FIG. 5. Representative GFP expression data from line #IV cells (upper panel) relative to wild-type control (lower panel).

FIG. 6. Flow cytometry data of cell line #M (ploidy analysis) relative to wild-type and #42061 parent controls.

FIG. 7. PCR detection of transgenes in plants produced from selected cell lines relative to donor and recipient parents and wild-type control.

FIG. 8. Progeny analysis of reciprocal crosses of line #K and wild type (Wt) plants.

FIG. 9. Progeny analysis of reciprocal crosses of line #III and wild type (Wt) plants.

FIG. 10. Progeny analysis of line #K plants after selfing.

FIG. 11. Sensitivity of parental plants (42061 and 138202) to spectinomycin and paromomycin.

FIG. 12. GFP expression in protoplasts of parental line #138202.

FIG. 13. GFP and GUS expression in line+8 (42061+138202) produced after selection on both spectinomycin and paromomycin relative to control.

FIG. 14. Chromosome karyotype analysis of +8 and #9 plants produced after cell fusion or transfer.

FIG. 15. Morphology of regenerated plants from selected +8 and #9 lines relative to parental control plants.

FIG. 16. Progeny analysis of cross ♀ #9 ♂ a Wt N. tabacum var. Samsun with selection relative to wild-type plants.

FIG. 17A. GFP reporter construct with lox sites to obtain a detectable phenotype in the presence of Cre recombinase;

FIG. 17B. GFP-positive corn callus cells indicating transfer of Cre recombinase.

### LISTING OF THE SEQUENCES

SEQ ID NO:1 gus forward primer

SEQ ID NO:2 gus reverse primer

SEQ ID NO:3 gfp forward primer

SEQ ID NO:4 gfp reverse primer

SEQ ID NO:5 npt2 forward primer

SEQ ID NO:6 npt2 reverse primer

SEQ ID NO:7 aadA forward primer

SEQ ID NO:8 aadA reverse primer

### DESCRIPTION

The present disclosure provides novel methods and compositions for transferring plastid-encoded and nuclear-encoded genetic traits, and/or cellular, cytoplasmic or nuclear components or expression products, between plant cells and tissues to create cells or plants with a desired genotype and/or combination of traits. Transformation of the plastid genome (“plastome”) is difficult or limited in many plant species. Therefore, it would be beneficial to have an efficient and effective technology for transferring or moving genetic material, e.g. as found in plastids, which may also be transformed or engineered through various molecular biology techniques, from one plant to another. Furthermore, movement of nuclear-encoded genetic material or other cellular components from one plant to another would also be useful, and novel methods to accomplish these objectives are also provided.

The present disclosure describes methods of cell-to-cell juxtaposition or contact and whole or partial transfer, exchange, or fusion of cellular components among a mixed population of plant cells of two or more different types, such as from two or more different parental plants, growing in vitro, for instance as a callus or cell suspension culture, which may be accompanied and aided by wounding of those cells or tissues in culture. Such a cellular transfer, exchange or fusion may result in a combination of traits from two different plant cells, or the creation of new traits or genotypes, via transfer, exchange or inclusion of one or more cytoplasmic or nuclear components from the other cell. Without being bound by theory, wounding of the plant cells, for instance by chopping with a razor blade, knife, or other sharp instrument, sonication, vortexing, shaking, blending, electroporation, or other means, is thought to create openings or pores in the plant cell wall that can allow for plasma membrane contact, exchange, or transfer between neighboring cells. The plasma membranes of cells in contact or close proximity may allow for transfer of genetic material or other cellular components between parental cells. Without being bound by theory, the plasma membranes may form a contiguous plasmalemma, thus allowing for whole or partial “cell fusion” or transfer or exchange of cellular components and/or genetic material (plastid and/or nuclear genetic material) between parent cells resulting in a product cell comprising a combination of cellular and/or genetic components or material from the two parental cells. According to some embodiments, an agent that promotes cell membrane fusion may also be utilized, such as the use of different osmoticums (e.g., polyethylene glycol (PEG), sugars, sugar alcohols, etc.), presence of high calcium (or other cation) concentration, higher pH, and/or other compounds and conditions that are known to promote cell membrane fusion in other methods. Such a mixed population of cells, containing the combined or product cells produced by transfer, exchange or fusion between the different cells of the mixture, may then be grown and regenerated, typically with screening or selection for a marker gene (transgenic or non-transgenic) present in one or another of the parental plant cells' genome, or by the creation of a new trait or marker expression. Plants grown or regenerated from these combined cells may then be identified, isolated or selected based on a novel assortment or combination of traits, such as a combination of genetic traits and/or markers, from the two or more parental cells, or by the creation of a new trait or marker expression.

Transfer of chloroplasts by protoplast fusion has been described (Sidorov et al., Planta 152:341-345, 1981; Sigeno et al., Plant Cell Rep 28:1633, 2009). But methods of protoplast isolation, fusion and plant regeneration are not developed for most commercial crops including corn, soybean, wheat, and others. Plastid movement between plants has also been reported by plant grafting (Thyssen et al. PNAS 107:2439-2443, 2012; Stegemann et al., PNAS 109:2434-2438, 2012). In these studies, scion and rootstock of different Nicotiana species with different nuclear and plastid selectable markers were used. After successful grafting, the grafted area of the stem was sliced and placed on selection media with selection agents for both chloroplast and nuclear markers. Plants with plastids of one original parent and nuclear genetic traits of the other parent could be regenerated.

However, the experiments described in Thyssen et al. and Stegemann et al. were limited to grafting of plant tissues. A similar approach of grafting plants was used for horizontal transfer of nuclear genome (Fuentes et al., Nature 511:232-235, 2014). In contrast, a cell transfer or combination method is described herein, involving a mixed population of cells from two or more parental types growing in vitro, for instance as callus or suspension, to promote intercellular combination, transfer or exchange of genetic and/or cellular components or traits. Such a mixed cell population, with cells of different species, varieties or genotypes in close contact or proximity, may undergo plasmalemma fusion or other active or passive mechanism to incorporate or transfer one or more portions of the cytoplasm, cellular organelles, such as plastids, and even the nucleus from another cell, or genetic material, expression products or other components thereof, which can be further promoted by wounding cells or clumps or clusters of cells in the mixture. Such a transfer or exchange between cells may effectively cause or allow for transfer of genetic material and/or other cellular components from donor cells of one genotype or genetic background (e.g. plastid or nuclear genetic background) to recipient cells of another genotype or genetic background. In contrast to the grafting experiments described for instance by Thyssen et al., which may be occurring through plasmodesmata, the present methods involve transfer, fusion or exchange between cells, such as between or among callus cells or cell suspension cultures, which would not have plasmodesmata. Indeed, the transfer of organelles and especially nuclei between cells as described herein, would likely not occur through plasmodesmata, since nuclei are too large to pass through plasmodesmata, even if formed and present between cells. Thus, unlike prior methods, the cell transfer or combination methods described herein do not require protoplasting, formation of plasmodesmata, nor successful grafting of differentiated plant tissues.

As described in the examples below, non-organized growing tissue (callus) from tobacco var. Samsun, having nuclear markers NPTII and GUS, and tobacco var. Petit Havana with plastid markers aadA and GFP were mixed together, wounded and placed for regeneration on selection medium with selection agents for the aadA and NPTII genes. Plants with aadA/GFP positive plastids and NPTII/GUS nuclear background were produced indicating cellular transfer between the two different parental cells. Molecular analysis confirmed the presence in such plants of all four genes. Morphology and ploidy level analysis confirmed the production of diploid plants similar to var. Samsun possessing transformed plastids from var. Petit Havana. Reciprocal crosses between plants produced by this method and wild-type tobacco plants confirmed that progeny plants had maternal inheritance of resistance to streptomycin/spectinomycin and expression of GFP, and had the nuclear-encoded NPTII combined with GUS expression.

This disclosure provides methods for producing a wounded mixed cell culture or population, and a composition comprising such a wounded mixed culture or population, comprising one or more combined or product cells that may comprise cellular components and/or genetic material from both parental cells or cell types. The mixed cell population may comprise two or more different parental genotypes, which may each have one or more unique or different transgenes, markers, recombination events, insertions, deletions, mutations, edits, etc. These methods can allow for effective transfer of genetic material or gene expression products between cells of different genotypes or genetic backgrounds. In certain embodiments, the plant cells are dicot plant cells, such as from tobacco, tomato, soybean, cotton, canola, alfalfa, sugar beets, Arabidopsis, or other fruits and vegetables. In other embodiments the plant cells may be from monocot plants, such as from corn, wheat, rice, sorghum, barley, or other cereal plants and vegetables. The cells may be from an in vitro grown cell culture, such as a cell suspension or a callus culture, which may be a regenerable callus culture. It is also possible that a donor parent, or a cell, callus or cell suspension from the donor parent, may be non-regenerable, but a cell, callus or cell suspension from the recipient parent may be regenerable, such that a cell produced by the present methods can be regenerated into a plant.

As used herein, a “parental cell(s)” or “parent cell(s)” refers to a cell, such as a cell suspension or callus cell, having one set of nuclear, mitochondrial and plastid genotype(s), although multiple plastid and/or mitochondrial genotypes may be present in the same cell due to the presence of multiple plastids and mitochondria per cell. A “parental cell” may be a donor cell or a recipient cell. A “parental plant” refers to a plant from which a parental cell is produced or derived.

As used herein, a “mixed population” refers to a mixture or combination of two or more different parental cells having a different genotype or genetic background, such as at least one transgene, marker, mutation, allele, insertion, deletion, edit or other genetic or sequence element in its nuclear, plastid and/or organellar genome(s) that is/are different between the two or more different parental cells. Methods for mutagenizing the nuclear, plastid and organellar genome(s) of plants and plant cells, and methods for introducing a targeted insertion, mutation or change into the plastid genome of a plant through recombination, and selecting for those mutations, insertions, etc., are known in the art. Similarly, methods for introducing a transgene into the nuclear or plastid genome of a plant or plant cell are also known in the art.

As used herein, a “donor cell” is a parental cell that provides a genetic element or trait to another parental cell (i.e., a recipient cell) in a cell transfer or combination method or experiment as provided herein. Indeed, a “recipient cell” is a parental cell that receives a genetic element or trait or cellular component from a donor cell in a method or experiment as provided herein. Typically, the recipient cell will not have the genetic element or trait transferred from the donor cell, or will not have a genetic element or trait that is the same as the genetic element and/or trait transferred from the donor cell, prior to performing the cell transfer or combination method or experiment. A “donor plant” is a plant from which a donor cell is produced or derived, and a “recipient plant” is a plant from which a recipient cell is produced or derived. A “genetic element” can include any sort of sequence or sequence variation or difference in a genome of a plant cell, which may give rise to a trait or phenotype in a plant.

As used herein, a “combined cell”, a “combined product cell” or a “product cell” is a cell produced by a method or experiment of the present disclosure that has a combination of genetic element(s) and/or trait(s), and/or a combination of cellular components or expression products, from the two or more parental cells as described (e.g., at least one genetic element, cellular component and/or trait from one parental cell and at least one genetic element, cellular component and/or trait from a different parental cell). In some embodiments, a “product cell” refers to a cell produced by a method or experiment of the present disclosure that has an edit or targeted (site-directed) insertion introduced by a site-specific nuclease expressed by a donor cell, or a site-specific nuclease expressed by a recipient cell in conjunction with a guide RNA expressed from donor cell. An “edit” refers to a change (e.g., insertion, deletion, substitution, inversion, etc.) in the nuclear genomic sequence of a resulting or product plant cell, and in a plant developed or regenerated from such a product plant cell, or a progeny plant thereof, and in a plant part or seed from any of the foregoing, relative to the corresponding genomic sequence of an otherwise identical plant cell or plant, such as a parental or recipient plant cell or plant, which was not been subjected to such “editing”. Such an edit may be within an intergenic region of a plant genome or a genic region of a plant genome, such as at or near a native gene or transgene (e.g., in an enhancer, promoter, splice site, coding sequence, exon, intron, 5′ or 3′ untranslated region (UTR), terminator, etc.) present in the recipient cell, to affect the expression and/or activity of such gene or transgene. To the extent that a recipient plant and a donor plant have different traits or phenotypes, a plant developed or regenerated from a product or combined cell, and progeny thereof, will often have traits and phenotypes including morphological and reproductive traits that are more similar or identical to the recipient plant due to a relatively minor genetic and/or cellular contribution of the donor cell being transferred to the recipient cell, and the combined product cell retaining most or all of the nuclear, mitochondrial and/or plastid genomes and/or cellular components of the recipient cell, with the exception of the genetic element(s), cellular component(s) and/or trait(s) transferred from the donor cell.

Wounding may be accomplished by methods known in the art. For instance, chopping or cutting of cells with a razor blade, knife or other sharp instrument, and wounding by sonication, have been found to be effective. Wounding may also be achieved by vortexing, shaking, blending, electroporation, or other mechanical means. Wounding preferably occurs after mixing of the two (or more) parental cells, but may also occur prior to mixing parental cells. Without being bound by theory, wounding of plant cells may create holes or pores in plant cell walls, and/or stimulate interaction of the two parental cell types, which may allow for additional areas of contact or juxtaposition of plasma membranes from two adjacent wounded cells. In the process of wounding or repair, a plant cell may take up the contents of another cell in the mixture. Without being bound by theory, the plasma membrane of two cells may interact or fuse, or the plasma membrane of one cell may allow the transfer of cellular components of another cell, producing a product or combined cell comprising at least a portion of the cytoplasm, organelle(s), nucleus, and/or genetic material from both of the original parental cells.

Once a wounded mixed cell culture has been produced, selection or screening for the presence of a desired genetic trait or marker, or a desired combination of genetic traits and/or markers, may be performed, during and/or after growth and regeneration of the mixed cell culture and/or plants or plant parts regenerated therefrom to select or screen for cells, plants or plant parts having at least part of the genetic material from both parental cell types. In certain embodiments, selection is imposed after production of the mixed cell culture, which may occur immediately after production of the mixed cell culture and/or later (e.g., even while the wounded population of cells is being prepared). Selection may occur, for example, by incorporation of an effective amount of a selective agent within one or more culture media.

In certain embodiments it may be desirable to utilize transgenic traits for selection or screening. Such traits may, for instance, include antibiotic or herbicide tolerance, such as resistance to kanamycin, streptomycin, spectinomycin, hygromycin, glyphosate, glufosinate, dicamba, etc. These traits may be plastid-encoded or nuclear-encoded. Other traits useful for selection or screening may include those which result in production of a visually detectable phenotype or product, such as GUS, GFP, or a carotenoid, such as phytoene, etc.

As used herein, “genome transfer,” “genetic transfer” or “gene transfer” refers to introduction of one or more nuclear genome- and/or plastid genome-encoded genetic traits and/or genes, such as all or part of the nuclear and/or plastid genome(s), from a donor plant cell into a recipient plant cell to form a combined product cell. Nuclear and/or plastid genome transfer may include the introduction of a portion of a nuclear and/or plastid DNA, a nuclear and/or plastid chromosome, or more than one nuclear and/or plastid DNA, including introduction of at least one complete organelle and/or at least one complete organellar, mitochondrial, plastid, and/or nuclear genome. Plastid genome transfer may include introduction of part or all of a chloroplast genome, and may result in heteroplastomic or homoplastomic cells. In some cases, a product cell may retain most or all of the cellular components and genome of one parental cell (recipient cell) and receive one or more cytoplasmic and/or genetic components from another parental cell (donor cell), such as one or more organelles and/or genomes of the donor parental cell, although a fused product cell may also lose one or more cellular, cytoplasmic and/or genetic components from a parental recipient cell in addition to gaining one or more cellular, cytoplasmic and/or genetic components from a parental donor cell.

As used herein, “wounding” refers to any treatment of cells that allows or promotes plasmalemma and/or cytoplasmic contact between different cells in a culture. For instance, wounding may occur by shaking, vortexing, sonication, cutting, and/or chopping of cells. Thus, when a cell wall is damaged or perturbed, for instance by chopping with a razor blade or by sonication, an opening or pore in a plant cell wall may be made, which may allow or promote the exchange of cellular material between the two cells.

Wounding a mixed population of cells growing in vitro can result in a “combined” product cell comprising a combination of one or more genomic, genetic and/or cellular component(s) from two or more parental cells, cell lines or cell types in the mixture. Without being bound by theory, contact or interaction between cells (e.g., plasma membranes (or plasmalemma) of adjacent cells), which may each be wounded cells, can provide for effective movement of genetic and/or cytoplasmic material from cells of, for instance, one genotype or genetic background to cells of another genotype or genetic background. The presence of organelles and/or genetic material from one or both (or more) parental lines or cells in a resulting product or combined cell may be promoted by application of selection pressure or screening or selecting for a marker or phenotype.

The term “transgene” refers to an exogenously introduced DNA molecule or construct that is incorporated into an organism's genome as a result of human intervention, such as by plant transformation methods. As used herein, the term “transgenic” refers to a material comprising a transgene or recombinant expression cassette or construct. For example, a “transgenic plant” refers to a plant comprising a transgene or recombinant expression cassette or construct in its genome, and a “transgenic trait” refers to a characteristic or phenotype caused, conveyed or conferred by the presence of a transgene or recombinant expression cassette or construct incorporated into the plant genome. As a result of such genomic alteration, the transgenic plant is something distinctly different from a related wild-type plant. According to many embodiments, a transgene may comprise a coding sequence or transcribable DNA sequence operably linked to a promoter, such as a plant-expressible promoter. A plant-expressible promoter may express in one or more plant cells, such as a parental, donor, recipient and/or product or combined cell according to the present disclosure. A plant-expressible promoter may be a constitutive promoter, a tissue-specific or tissue-preferred promoter, a developmental stage promoter, or an inducible promoter. According to many embodiments, the transcribable DNA sequence or coding sequence of the transgene may encode a RNA or protein of interest, such as a structural protein, enzyme, RNA suppression element or guide RNA for a site-specific nuclease. According to some embodiments, a coding sequence of a transgene may comprise a coding sequence of a marker gene, which may be present in the nuclear or plastid genome. The marker gene may be a selectable marker gene or a screenable marker gene as further described herein. According to some embodiments, a coding sequence of a transgene may encode a site-specific nuclease.

As used herein and according to its commonly understood meaning, a “control” means an experimental control designed for comparison purposes, which is typically similar to an experimental or test subject except for the one or more differences or modifications being tested or studied. For example, a control plant may be a plant of the same or similar type as the experimental or test plant having one or more modifications of interest (e.g., a transgene, mutation, edit, etc.) that does not contain the modification(s) present in the experimental plant.

### Transgenic Plants

An aspect of the invention includes transgenic plant cells, transgenic plant tissues, transgenic plants, and transgenic seeds that comprise recombinant DNA molecules in a novel assortment, i.e. in a combination distinct from that found in any previously existing parental plant line, or plant cell line. These cells, tissues, plants, and seeds comprising the recombinant DNA molecules, transgenes, constructs, cassettes, etc., may exhibit tolerance to a selection agent, such as one or more herbicides or antibiotics, or provide a screenable marker or another phenotype or trait of interest, such as an agronomic trait of interest. According to some embodiments, a plant cell used in a cell transfer method or experiment of the present disclosure may be a transgenic plant cell, which may be further derived from a transgenic plant.

Suitable methods for transformation of plant cells for use with the current cell transfer methods include any method by which DNA can be introduced into a cell (for example, where a recombinant DNA construct is stably integrated into a plant chromosome). Methods of plant transformation are known in the art. Methods for introducing a recombinant DNA construct into plants may include bacterially-mediated (or Agrobacterium-mediated) transformation or particle-bombardment techniques for transformation, both of which are well known to those of skill in the art. Another method that may be used for introducing a recombinant DNA construct into plants is insertion of a recombinant DNA construct into a plant genome at a pre-determined site by methods of site-directed integration. Site-directed integration may be accomplished by any method known in the art, for example, by use of zinc-finger nucleases, engineered or native meganucleases, TALE-endonucleases, or an RNA-guided endonuclease (for example, a CRISPR/Cas9 system) in combination with a template DNA for making the genomic insertion at a desired target site. Thus site-directed integration may be used to introduce a transgene at a desired location in the genome. Methods for culturing explants and plant parts, as well as methods for selecting and regenerating plants in culture, are also known in the art.

Transgenic plants can be developed or regenerated from a transformed plant cell, tissue or plant part by any known culturing methods for plant cells, tissues or explants. A transgenic plant homozygous with respect to a transgene (that is, two allelic copies of the transgene) can be obtained by self-pollinating (selfing) a transgenic plant that contains a single transgene allele with itself, for example an R0 plant, to produce R1 seed. Transgenic offspring, such as plants grown from R1 seed, can be tested for zygosity using any known zygosity assay, such as by using a SNP assay, DNA sequencing, thermal amplification or PCR, and/or Southern blotting that allows for the distinction between heterozygotes, homozygotes and wild type.

Plants and progeny that contain a novel combination of traits as provided herein may be used with any breeding methods that are commonly known in the art. In plant lines comprising two or more transgenic traits, the transgenic traits may be genetically linked or independently segregating, and plant lines comprising three or more transgenic traits may comprise traits that are both linked and independently segregating. Methods for breeding or crossing plants that are commonly used for different traits and crops are known to those of skill in the art. For example, introgression of a transgenic trait, allele or genetic locus into a plant genotype can be achieved by backcross conversion. A plant genotype into which a transgenic trait has been introgressed may be referred to as a backcross converted genotype, line, inbred, or hybrid. Similarly, a plant genotype lacking the desired transgenic trait may be referred to as an unconverted genotype, line, inbred, or hybrid.

Aspects of the present disclosure may be used in breeding or introgression efforts as a replacement for crossing plants through sexual reproduction to allow for a combination of genetic traits and/or cellular components in combined product cells, which may be developed or regenerated into plants having a desired combination or introduction of traits. Such plants may be identified or selected based on the presence of one or more markers, traits or phenotypes. To confirm the presence of the transgene(s), mutation(s) or other trait(s) in a plant, plant part or seed or progeny thereof, such as a plant regenerated from a combined product cell as provided herein, or a plant part, seed or progeny thereof, a variety of assays may be performed and used. Such assays can include, for example, molecular biology assays, such as Southern and northern blotting, PCR, and DNA sequencing; biochemical assays, such as detecting the presence of a protein product, for example, by immunological means (ELISAs and western blots) or by enzymatic function; plant part assays, such as leaf or root assays; and also by analyzing a phenotype or trait of the whole plant.

### Gene Editing and Recombination

The ability to transfer cytoplasmic materials and components between cells in a mixed cell culture according to present methods provides the potential to transfer RNA, protein and/or other molecules or factors present in the cytoplasm or cytosol of one plant cell to another plant cell. These molecules from a donor cell may be transferred to the cytoplasm of a recipient cell without changing or inserting into the genomic DNA of the recipient cell. Thus, RNA and/or protein may be transferred from a donor cell to a recipient cell according to present methods and exert an activity, effect or change on the recipient cell. The transferred RNA, protein or other molecule may be present only transiently, since the gene encoding the RNA or protein may not be transferred to the recipient cell and other molecules may not be made or produced by the recipient cell. Thus, the RNA, protein or other molecule may only be present in the recipient cell for a limited time depending on its starting concentration in the recipient cell following transfer and its stability or half-life in the recipient cell.

As demonstrated in Example 3 below, a mixed population of corn cells was generated comprising one group of cells expressing a Cre recombinase enzyme and another group of cells comprising a GFP reporter construct with lox sites flanking an intervening sequence that will express GFP when the intervening sequence is excised by the Cre enzyme acting on the lox sites. In this experiment, positive GFP clones were generated after wounding the cells indicating the generation of recombination events in cells due to the transfer of Cre recombinase from a donor cell to a recipient cell. This result could be explained by transfer of the Cre-expressing transgene from the donor cell to the recipient cell, which may then be transiently expressed in the recipient cell or stably integrated into the recipient cell genome. Alternatively or additionally, the Cre recombinase enzyme expressed from the transgene in the donor cell may have been transferred to the recipient cell where it acted on the lox sites in the recipient cell, without the Cre-expressing transgene being transferred to the recipient cell.

The ability to deliver RNA and/or protein to a recipient cell, without transforming, integrating or incorporating a transgene(s) encoding the RNA and/or protein into the recipient cell genome, makes it possible to make changes to a non-transgenic recipient cell genome (i.e., without transforming the genome of the recipient cell with transgene) by delivering RNA and/or protein from the donor cell. Similar to Cre recombinase, other enzymes that can be expressed in a donor cell and delivered to a recipient cell to make changes to the recipient cell DNA. According to some embodiments, a site-specific nuclease, such as a zinc-finger nuclease, a meganuclease, an RNA-guided nuclease, a TALE-nuclease, a recombinase, a transposase, or any combination thereof, may be expressed in a donor cell and become transferred to a recipient cell via a method of the present disclosure, which may involve wounding the cells of a mixture comprising the donor and recipient cells. In some embodiments, the RNA-guided nuclease is a CRISPR associated nuclease (non-limiting examples of CRISPR associated nucleases include, for example, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof). In some embodiments, the donor cell expresses both the RNA-guided nuclease and the guide RNA which are delivered to a recipient cell to make changes to the recipient cell DNA. In some embodiments, the donor cell expresses the RNA-guided nuclease which is delivered to a recipient cell expressing the guide RNA which complexes with the RNA-guided nuclease to make changes to the recipient cell DNA. In some embodiments, the donor cell expresses the guide RNA which is delivered to a recipient cell expressing the RNA-guided nuclease which complexes with the guide RNA to make changes to the recipient cell DNA. In some embodiments the donor cell may further comprise a donor DNA sequence. In some embodiments the donor DNA sequence is a template for templated editing. In other embodiments the donor DNA sequence comprises a transgene. An edited product cell generated by transfer of a site specific nuclease fom a donor cell to a recipient cell may be regenerated into a plant having the edit in its genome, and progeny plants, plant parts and seeds can also be derived from the regenerated plant. In many embodiments, plants regenerated from the edited product cell may be genetically and phenotypically similar to the plants from which the recipient cell was derived except for any trait(s) and/or phenotype(s) that are caused by the genomic edit or mutation.

According to many of these embodiments, a method is provided for editing a plant cell comprising: mixing a first plant cell culture and a second plant cell culture to obtain a mixed cell culture, wherein one or more cells of the first plant cell culture comprise a recombinant DNA transgene comprising a sequence encoding a site-specific nuclease operably linked to a first promoter; and wounding the cells of the mixed cell culture to produce at least one edited product cell having an edit or mutation introduced in its genome by the site-specific nuclease. Such methods may also comprise screening or selecting for the at least one edited product cell, or a progeny cell thereof, or a plant developed or regenerated from the at least one edited product cell, or a progeny cell thereof, having the edit or mutation, which may be based on a molecular assay or a trait or phenotype produced by the edit or mutation and present in a plant developed or regenerated from the edited product cell or a progeny cell thereof, or present in a progeny plant, plant part or seed thereof. In these methods, the first and second plant cell cultures can be callus cultures or cell suspension cultures. These methods may further comprise regenerating a plant from the mixed cell culture and/or the at least one edited product cell, or at least one progeny cell thereof. The plant cells used in these methods may be monocot or dicot plant cells.

According to some embodiments, the one or more cells of the first plant cell culture in these methods may further comprise a first recombinant DNA construct comprising a first transcribable DNA sequence encoding a guide RNA molecule operably linked to a promoter. According to some embodiments, the one or more cells of the first plant cell culture in these methods may further comprise a second recombinant DNA construct comprising a second transcribable DNA sequence encoding a donor template molecule operably linked to a promoter. According to some embodiments, one or more cells of the second plant cell culture in these methods may further comprise a first recombinant DNA construct comprising a first transcribable DNA sequence encoding a guide RNA molecule operably linked to a promoter. According to some embodiments, one or more cells of the second plant cell culture in these methods may further comprise a second recombinant DNA construct comprising a second transcribable DNA sequence encoding a donor template molecule operably linked to a promoter.

Further provided are edited plant cells produced by these methods, and progeny cells thereof, which may be monocot or dicot plant cells, and which may each be further developed or regenerated into an edited plant. A seed or plant part of a developed or regenerated plant, or a progeny plant thereof, is also provided. In addition, the mixed cell culture of plant cells produced by these methods, which may be a wounded mixed cell culture, are further provided.

A site-specific nuclease provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp 1 recombinase. According to some embodiments, a Cre recombinase or a Gin recombinase may be tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of a Cas9 or a Cpf1. According to other embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 enzyme. For RNA-guided endonucleases, a guide RNA (gRNA) molecule may be further provided to direct the endonuclease to a target site in the genome of the plant via base-pairing or hybridization to cause a DSB or nick at or near the target site. The gRNA may be transformed or introduced into a plant cell or tissue as a gRNA molecule, or as a recombinant DNA molecule, construct or vector comprising a transcribable DNA sequence encoding the guide RNA operably linked to a promoter or plant-expressible promoter. The promoter may be a constitutive promoter, a tissue-specific or tissue-preferred promoter, a developmental stage promoter, or an inducible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such genomic locus to knockout or knockdown expression of the gene via repair of the DSB or nick, which may lead to the creation of a mutation or insertion of a sequence at the site of the DSB or nick, through cellular repair mechanisms, which may be guided by a donor template molecule.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp 1 recombinase. In an aspect, a Cre recombinase or a Gin recombinase provided herein may be tethered to a zinc-finger DNA binding domain. The Flp-FRT site-directed recombination system may come from the 2μ plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick.

Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, I-AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a gene. In another aspect, a meganuclease provided herein is capable of generating a targeted DSB.

TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also refers to one or both members of a pair of TALENs that work together to cleave DNA at the same site.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122.; Cermak et al., Nucleic Acids Research (2011). 39:e82; and tale-nt.cac.cornell.edu/about. In another aspect, a TALEN provided herein is capable of generating a targeted DSB.

According to some embodiments, a donor template may also be expressed by the donor cell and delivered to the recipient cell to serve as a template for the desired edit generated following the introduction of a double-stranded break (DSB) or nick in the recipient cell genome by the site-specific nuclease. Alternatively, a donor template may be expressed by the recipient cell. Similarly for RNA-guided nuclease, a transcribable DNA sequence or transgene expressing a guide RNA (gRNA) may also be present and expressed in the donor cell and delivered to the recipient cell to serve as a guide RNA for the RNA-guided nuclease to direct the RNA-guided nuclease to make a double-stranded break (DSB) or nick at the desired locus or target site in the recipient cell genome. Alternatively, a guide RNA (gRNA) may be expressed by the recipient cell. According to further embodiments, (i) a site-specific nuclease, a guide RNA and a donor template may all be expressed by the donor cell and become transferred to a recipient cell, or (ii) a site-specific nuclease and/or a guide RNA may be expressed by the donor cell and become transferred to a recipient cell, and a donor template may be optionally expressed in the recipient cell, or (iii) a site-specific nuclease and/or a donor template may be expressed by the donor cell and become transferred to a recipient cell, and a guide RNA may be optionally expressed in the recipient cell, or (iv) a guide RNA and/or a donor template may be expressed by the donor cell and become transferred to a recipient cell, and a site-specific nuclease may be expressed in the recipient cell, in each case (i), (ii), (iii) or (iv) to make a double-stranded break (DSB) or nick at the desired locus or target site in the recipient cell genome to give rise to a templated or non-templated edit or mutation a the desired location in the genome of the recipient cell.

Any site or locus within the genome of a plant may potentially be chosen for making a genomic edit (or gene edit) or site-directed integration of a transgene, construct or transcribable DNA sequence. For genome editing and site-directed integration, a double-strand break (DSB) or nick may first be made at a selected genomic locus with a site-specific nuclease, such as, for example, a zinc-finger nuclease (ZFN), an engineered or native meganuclease, a TALE-endonuclease, or an RNA-guided endonuclease (e.g., Cas9 or Cpf1). Any method known in the art for site-directed integration may be used. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct or sequence may be achieved if the transgene, transcribable DNA sequence, construct or sequence is located in the insertion sequence of the donor template.

The introduction of a DSB or nick may also be used to introduce targeted mutations in the genome of a plant. According to this approach, mutations, such as deletions, insertions, inversions and/or substitutions may be introduced at a target site via imperfect repair of the DSB or nick to produce a knock-out or knock-down of a gene. Such mutations may be generated by imperfect repair of the targeted locus even without the use of a donor template molecule. A “knock-out” of a gene may be achieved by inducing a DSB or nick at or near the endogenous locus of the gene that results in non-expression of the protein or expression of a non-functional protein, whereas a “knock-down” of a gene may be achieved in a similar manner by inducing a DSB or nick at or near the endogenous locus of the gene that is repaired imperfectly at a site that does not affect the coding sequence of the gene in a manner that would eliminate the function of the encoded protein. For example, the site of the DSB or nick within the endogenous locus may be in the upstream or 5′ region of the gene (e.g., a promoter and/or enhancer sequence) to affect or reduce its level of expression. Similarly, such targeted knock-out or knock-down mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out mutations of a gene may be achieved by deleting or inverting at least a portion of the gene or by introducing a frame shift or premature stop codon into the coding sequence of the gene. A deletion of a portion of a gene may also be introduced by generating DSBs or nicks at two target sites and causing a deletion of the intervening target region flanked by the target sites.

As used herein, a “donor molecule”, “donor template”, or “donor template molecule” (collectively a “donor template”), which may be a recombinant polynucleotide, DNA or RNA donor template, is defined as a nucleic acid molecule having a nucleic acid template or insertion sequence for site-directed, targeted insertion or recombination into the genome of a plant cell via repair of a nick or double-stranded DNA break in the genome of a plant cell. For example, a “donor template” may be used for site-directed integration of a transgene or suppression construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A “donor template” may be a single-stranded or double-stranded DNA or RNA molecule or plasmid. An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template.

An insertion sequence of a donor template may comprise one or more genes or sequences that each encode a transcribed non-coding RNA or mRNA sequence and/or a translated protein sequence. A transcribed sequence or gene of a donor template may encode a protein or a non-coding RNA molecule. An insertion sequence of a donor template may comprise a polynucleotide sequence that does not comprise a functional gene or an entire gene sequence (e.g., the donor template may simply comprise regulatory sequences, such as a promoter sequence, or only a portion of a gene or coding sequence), or may not contain any identifiable gene expression elements or any actively transcribed gene sequence. Further, the donor template may be linear or circular, and may be single-stranded or double-stranded. A donor template may be delivered to a cell as a RNA molecule expressed from a transgene. A donor template may also be delivered to the cell as a naked nucleic acid (e.g., via particle bombardment), as a complex with one or more delivery agents (e.g., liposomes, proteins, poloxamers, T-strand encapsulated with proteins, etc.), or contained in a bacterial or viral delivery vehicle, such as, for example, Agrobacterium tumefaciens or a geminivirus, respectively. An insertion sequence of a donor template provided herein may comprise a transcribable DNA sequence that may be transcribed into an RNA molecule, which may be non-coding and may or may not be operably linked to a promoter and/or other regulatory sequence.

According to some embodiments, a donor template may not comprise an insertion sequence, and instead comprise one or more homology sequences that include(s) one or more mutations, such as an insertion, deletion, substitution, etc., relative to the genomic sequence at a target site within the genome of a plant, such as at or near a gene within the genome of a plant. Alternatively, a donor template may comprise an insertion sequence that does not comprise a coding or transcribable DNA sequence, wherein the insertion sequence is used to introduce one or more mutations into a target site within the genome of a plant, such as at or near a gene within the genome of a plant.

A donor template provided herein may comprise at least one, at least two, at least three, at least four, at least five, at least six, at least seven, at least eight, at least nine, or at least ten genes or transcribable DNA sequences. Alternatively, a donor template may comprise no genes. Without being limiting, a gene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal. The leader, enhancer, and/or promoter may be operably linked to a gene or transcribable DNA sequence encoding a non-coding RNA, a guide RNA, an mRNA and/or protein.

According to present embodiments, a portion of a recombinant donor template polynucleotide molecule (i.e., an insertion sequence) may be inserted or integrated at a desired site or locus within the plant genome. The insertion sequence of the donor template may comprise a transgene or construct, such as a transgene or transcribable DNA sequence encoding a non-coding RNA molecule that targets an endogenous gene for suppression. The donor template may also have one or two homology arms flanking the insertion sequence to promote the targeted insertion event through homologous recombination and/or homology-directed repair. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant cell. Thus, a plant cell may comprise a recombinant DNA molecule encoding a donor template for site-directed or targeted integration of a transgene or construct, such as a transgene or transcribable DNA sequence encoding a non-coding RNA molecule that targets an endogenous gene for suppression, into the genome of a plant.

As used herein, a “target site” for genome editing or site-directed integration refers to the location of a polynucleotide sequence within a plant genome that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for a RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further below). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by another site-specific nuclease that may not be guided by a non-coding RNA molecule, such as a meganuclease, zinc finger nuclease (ZFN), or a transcription activator-like effector nuclease (TALEN), to introduce a double stranded break (or single-stranded nick) into the polynucleotide sequence and/or its complementary DNA strand.

As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

### EXAMPLES

The following examples are included to demonstrate certain embodiments of the present disclosure. It should be appreciated by those of skill in the art that these examples that follow represent techniques and approaches that may be used in the practice of present methods and embodiments. However, those of skill in the art should, in light of the present disclosure, appreciate that many modifications, changes and substitutions may be made to the specific embodiments disclosed herein to obtain a similar results without departing from the spirit and scope of the present disclosure.

### Example 1: Plastid Transfer from a Donor Line to a Recipient Line in Tobacco by Cell Fusion

### A. Establishment of Donor and Recipient Lines for Plastid Transfer

A plastid donor line Nicotiana tabacum var. Petit Havana (line #30125) was established as described (see Sidorov et al., Plant Journal, 19: 209-216, 1999) with a recombinant DNA construct in the plastid genome containing an aadA gene (conferring spectinomycin/streptomycin resistance) and a GFP marker gene.

A plastid recipient line N. tabacum var. Samsun (line #42061) was established by transforming its nuclear genome with a recombinant DNA construct in the nuclear genome containing an NPTII gene (conferring kanamycin or paromomycin resistance) and a GUS marker gene, via Agrobacterium-mediated transformation.

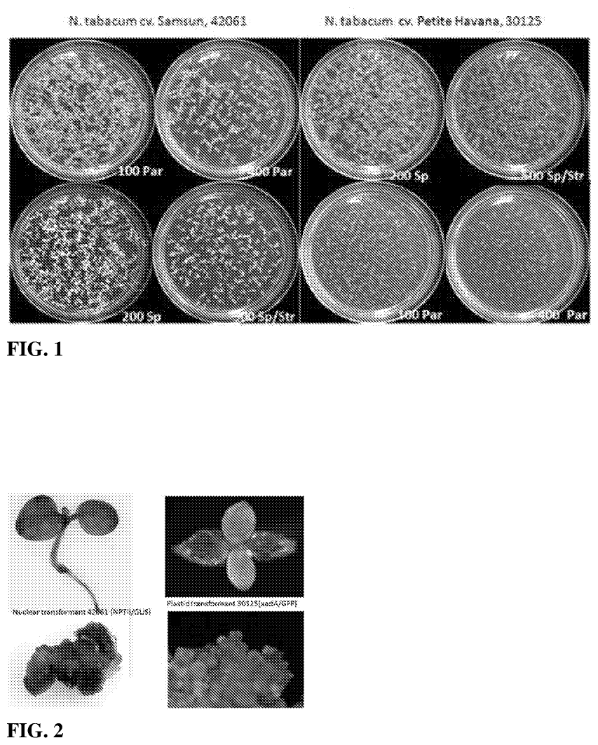

Seeds of plastid transformant line #30125 (aadA/GFP) and nuclear transformant line #42061 (NPTII/GUS) were germinated on media with the corresponding antibiotic selections and checked for expression of the resistance genes, i.e., aadA or NPTII (FIG. 1), and the screenable marker genes, i.e. GFP or GUS (FIG. 2)

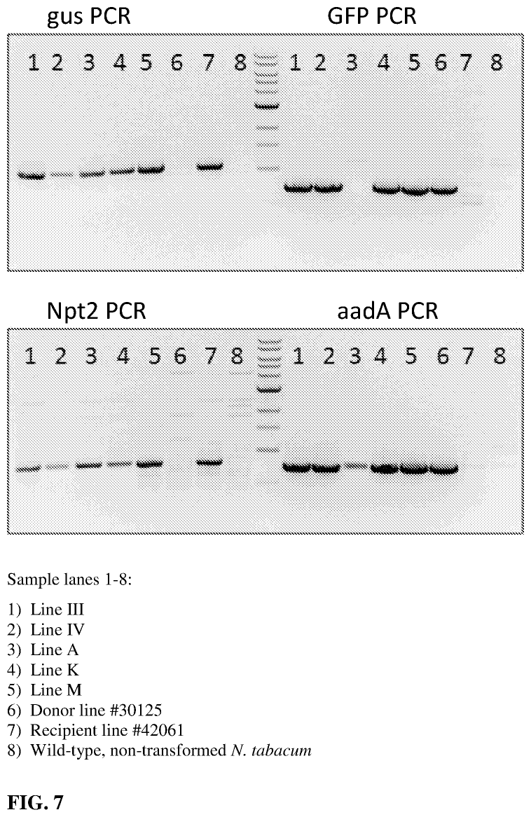

As shown in FIG. 1, germinated seeds of line #30125 were resistant to spectinomycin (labeled as Sp) or spectinomycin plus streptomycin (labeled as Sp/Str) but were sensitive to paromomycin (labeled as Par). Germinated seeds of line #42061 were resistant to paromomycin, but were sensitive to Sp or Sp/Str. Callus cells produced from these lines were also resistant to the corresponding antibiotics. It was also confirmed that selected plants and callus also comprised GUS or GFP respectively as shown in FIG. 7 (see below).

### B. Culture Conditions for Plastid Transfer

Seeds of both lines were sterilized with 5% commercial bleach and germinated on MS (Murashige and Skoog, 1962) medium without plant growth regulators. Callus of both donor and recipient lines were induced on MS medium with 1 mg/L BA (6-benzylaminopurine) and 1 mg/L NAA (1-Naphthaleneacetic acid), and MS medium with 1 mg/L BA and 0.1 mg/L NAA was subsequently used for regeneration of plants.

It was found that callus was less sensitive to the tested antibiotics than germinating seeds or plants. For instance, callus from line #30125 was inhibited at 400 mg/L Par and higher concentrations. Therefore, selection with callus cells was conducted on culture medium with 400-500 mg/L Par+400-500 mg/L Sp.

### C. Production of Wounded Mixed Callus Culture and Cell Fusion

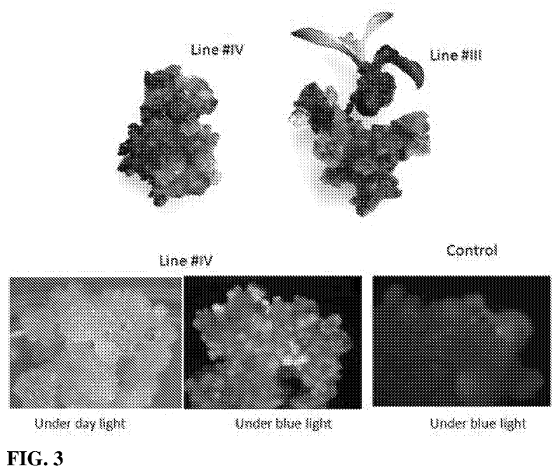

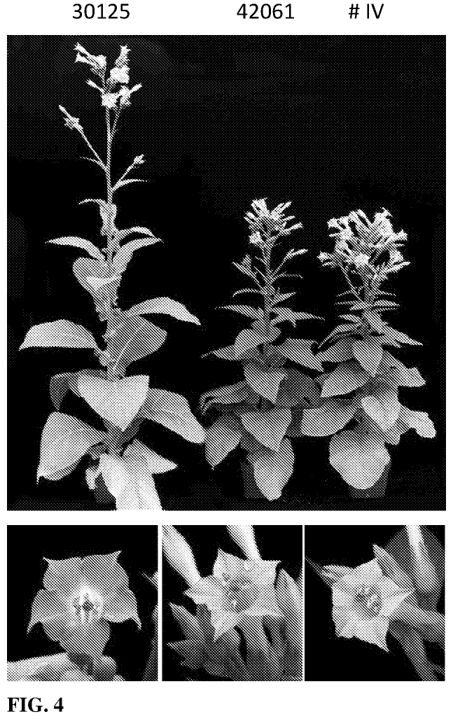

Callus induced from both donor and recipient lines were mixed together and chopped into fine pieces with a razor blade. This procedure produced a wounded mixed cell culture. A compact clump of the callus mixture was placed on selection medium with both Par and Sp (400 mg/l Par+400 mg/l Sp). In this experiment, four green cell lines having resistance to both Par and Sp were selected. The four selected cell lines, named #III, #IV, #K and #M, were also positive for both GFP and GUS staining. For example, expression of GUS in the #III and #IV cell lines, and expression of GFP in the #IV cell line, are shown in FIG. 3. The resistance to both Par and Sp and the presence of the GFP and GUS markers indicate that the selected cells are fused product cells with a combination of traits from both parental cells (donor and recipient). As further detailed below, phenotypically normal plants were regenerated from these four selected cell lines. The regenerated plants were fertile and had the combination of traits present in the cell lines including Sp/Par resistance and GUS/GFP expression. These traits were also retained and observed in progeny plants grown from seed.

In one experiment, a chlorophyll deficient cell line (line #A) resistant to high concentration of paromomycin and spectinomycin was also isolated. Formation of green callus from line #A was identified after several subcultures and later normal plants were regenerated. This line however did not have GFP expression and was spectinomycin-resistant but sensitive to streptomycin (data not shown).

D. Analysis of Selected Plants from Cell Combination or Transfer

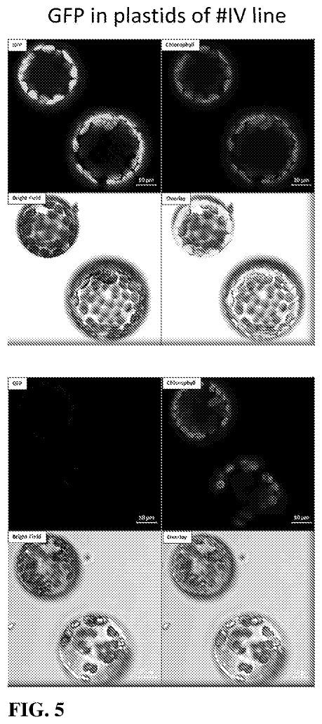

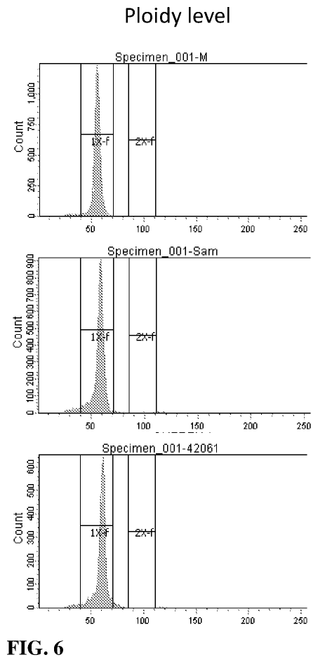

Plants regenerated from all selected cell lines and grown to maturity in the growth chamber had normal morphology that was similar to recipient line #42061 (see FIG. 4 for plants regenerated from line #IV). Confocal microscopy was used to confirm GFP expression in plastids of isolated mesophyll protoplasts from plants regenerated from the selected lines. Representative GFP expression in isolated protoplasts of line #IV is shown in FIG. 5, indicating that plastid-encoded genetic traits were transferred from donor cells. Flow cytometry of isolated protoplasts for analysis of ploidy level of four produced lines indicated that there was no difference in the amount of DNA among samples. Representative data from the flow cytometry experiment for line #M cells is shown in FIG. 6 in comparison to parent and wild type controls.

E. Molecular Analysis of Chloroplast in Produced Events after Cell Fusion

Fresh leaf materials were collected from individual plants regenerated from the selected #III, #IV, #K and #M lines, in addition to individual donor, recipient and wild-type plants, in 1.5-ml Eppendorf tubes and ground with a micropestle according to a known method (see Wang et al., NAR 21:4153-4154, 1993). Briefly, 10 μl 0.5 N NaOH was added to every mg of tissue and samples were ground with a micropestle until no large pieces of tissue were left. 2 μl of each sample was transferred quickly to a new tube containing 100 μl of 100 mM Tris pH 8.0, and mixed well. Samples were denatured for 5 minutes on a PCR machine at 99° C. and stored on ice. One μl of samples were directly used in 25 μl PCR reaction. The following PCR program was used in this experiment: 94° C. denature for 30 seconds, 56° C. annealing for 30 seconds, 72° C. elongation for 30 seconds, 35 cycles. The Q5® Hot Start High-Fidelity DNA Polymerase (NEB cat #M0493S) was used for all PCR.

The following gene specific primers were used for gus, GFP, aadA, and nptII gene detection:

Gus PCR primers: Amplifies a 1067 bp gus fragment
(gus forward)
(SEQ ID NO: 1)
5′ AAGACTGTAACCACGCGTCTG 3′
(gus reverse)
(SEQ ID NO: 2)
5′ ATTCCATACCTGTTCACCGAC 3′
GFP primers: Amplifies a 741 bp fragment
(gfp forward)
SEQ ID NO: 3)
5′ ATGTCACCACAAACAGAGGCC 3′
(gfp reverse)
SEQ ID NO: 4)
5′ TCATTATTTGTAGAGCTCATCCATGC 3′
Npt2 primers: Amplifies a 790 bp fragment
(npt2 forward)
(SEQ ID NO: 5)
5′GCATGATTGAACAAGATGGATTGCAC 3′
(npt2 reverse)
(SEQ ID NO: 6)
5′ GAACTCGTCAAGAAGGCGATAGAAGG 3′
aadA primers Amplifies 763 bp aadA fragment
(aadA forward)
(SEQ ID NO: 7)
5′ CGAAGTATCGACTCAACTATCAGAG 3′
(aadA reverse)
SEQ ID NO: 8)
5′ GACTACCTTGGTGATCTCGCCTTTC 3′

Molecular analysis demonstrated the presence of all four genes, i.e., nptII, aadA, uidA (gus), and GFP, in selected #III, #IV, #K and #M lines, in contrast to donor and recipient lines and WT plants, as shown in FIG. 7. This data indicates that recipient cells received the plastid-encoded traits from the donor lines.

### F. Genetic Analysis of Produced Events

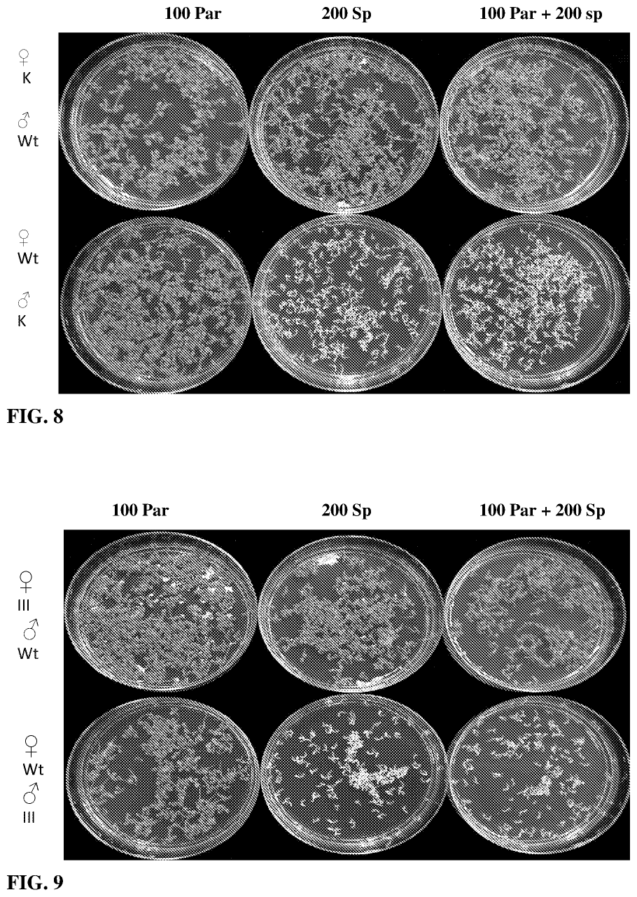

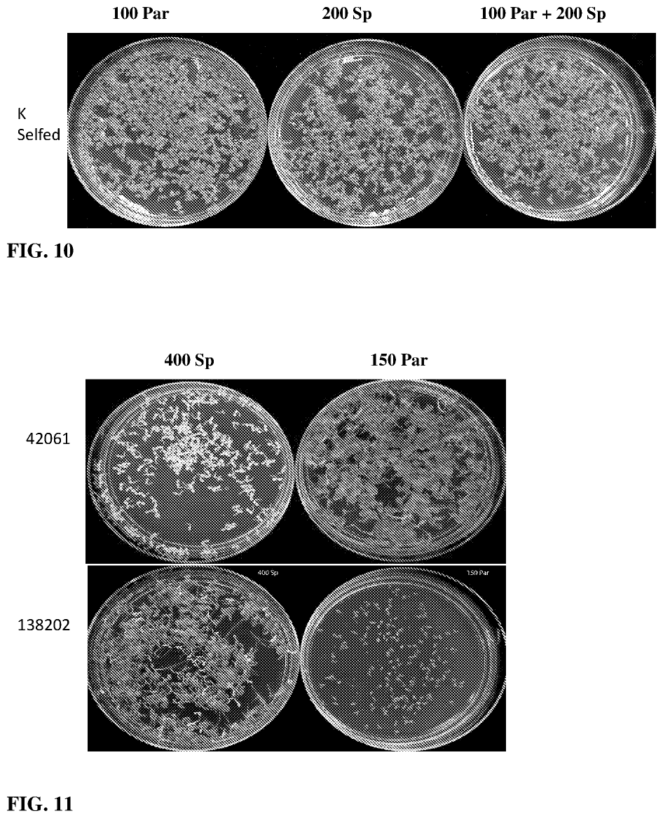

Genetic analysis of five plants produced from the above selected cell lines was conducted. Plants from the selected #III and #K selected lines were crossed reciprocally with wild type tobacco plants, and progeny plants were screened for resistance to antibiotics (FIGS. 8-9). Genetic analysis of selfed progenies of the #K selected cell lines was also performed (FIG. 10). These analyses confirmed Mendelian nuclear inheritance of the NPTII/GUS genes and maternal inheritance of the aadA/GFP genes. Thus, the present cell fusion method (using two different selection markers, i.e., one located in the donor's plastome and the other one located in recipient's nuclear genome) can be efficiently used for transferring of a plastid genome from donor to recipient cells and plants.

### Example 2: Nuclear Gene Transfer by Cell Transfer in Tobacco

Two transgenic tobacco plant lines were generated to demonstrate transfer of nuclear DNA by cell fusion in wounded mixed plant cell cultures. N. tabacum var. Samsun line #42061 was established by transforming its nuclear genome with a recombinant DNA construct comprising a NPTII gene (conferring Paromomycin resistance) and a GUS marker gene, and an EPSPS gene which provides resistance to glyphosate. N. tabacum var. Petit Havana line #138202 was established by transforming its nuclear genome with a recombinant DNA construct containing an aadA gene (conferring streptomycin and spectinomycin resistance), and both GFP and GUS marker genes.

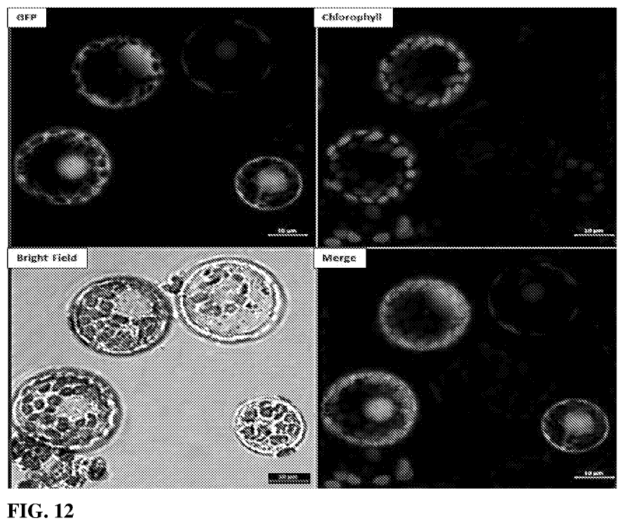

Plants transformed with these transgenes were checked to confirm the expression of their corresponding resistance gene as shown in FIG. 11. Plants of line #138202 were checked for GFP expression as shown in FIG. 12. GFP was localized in nuclei and cytoplasm. Established plants were checked for the corresponding resistance gene expression as shown in FIG. 11. Plants of line #138202 were checked for GFP expression, and GFP was found localized in nuclei and cytoplasm as shown in FIG. 12.

### A. Cell Wounding for Nuclear Gene Transfer.

Studies were conducted for transfer of nuclear traits by cell combination, fusion or transfer. About 4-5 g of callus from each parent cell line were mixed together, wounded thoroughly by chopping with a razor blade, and placed as compact mixture of callus on selection medium with both spectinomycin and paromomycin. Two lines resistant to both paromomycin and spectinomycin were selected and isolated as combined or fused product cells having a combination of genetic traits from both of the parental cell lines.

B. Analysis of Selected Plants from Cell Combination or Transfer.

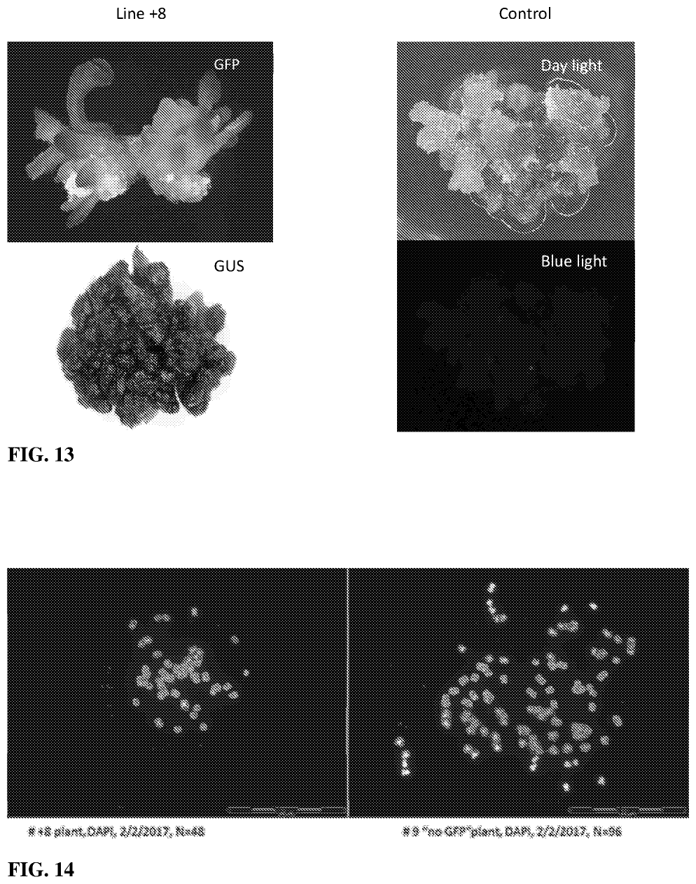

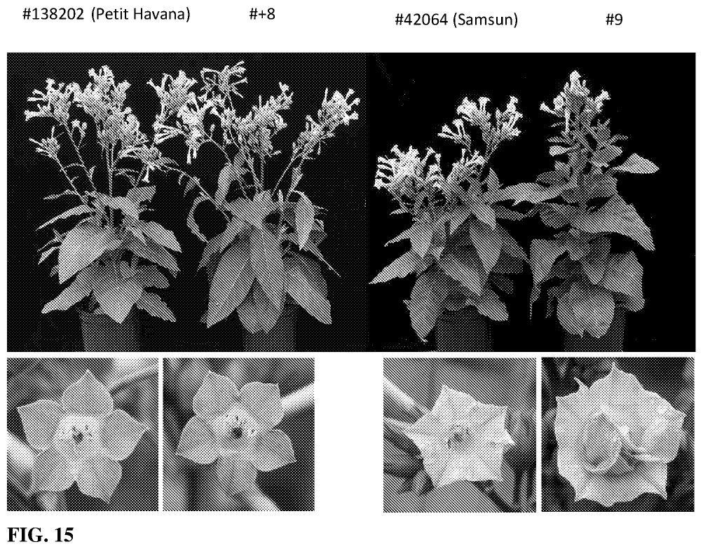

Selected line # +8 was GFP and GUS positive as shown in FIG. 13, while another selected line #9 was GUS positive but GFP negative. Root tips of regenerated plants were used for analysis of chromosome karyotype. Line # +8 had 49 chromosomes similar to wild type Nicotiana, indicating possible transfer of a portion of the nuclear plant genome. However, DAPI staining of the #9 line (GFP negative) indicated a double amount of chromosomes (about 96 chromosomes) as shown in FIG. 14, indicating possible production of an allopolyploid plant from the #9 line. The karyotyping results indicate that plants regenerated from # +8 line appeared may contain a limited amount of genetic material from the donor (#42061), while plants regenerated from line #9 contained much or all of the nuclear genome from both parents.

Given the ability to select for and detect the presence of markers from two different parental cells in a single plant cell, protoplast or plant, it is concluded that the cell transfer or combination method described herein using selection and/or detection markers from one or more parental plant nuclear genome(s) can be used for transferring nuclear genes between plant cells and plants. FIG. 15 shows that the morphology of the regenerated # +8 plant is similar to that of parental line #139202. However, the plant from #9 is more similar to #42061, but is different in that it has larger and thicker leaves and larger double flowers. Most stamens of these regenerated plants were converted to petals and were non-functional. Carpels of these plants were thicker, but flowers of these plants could be pollinated.

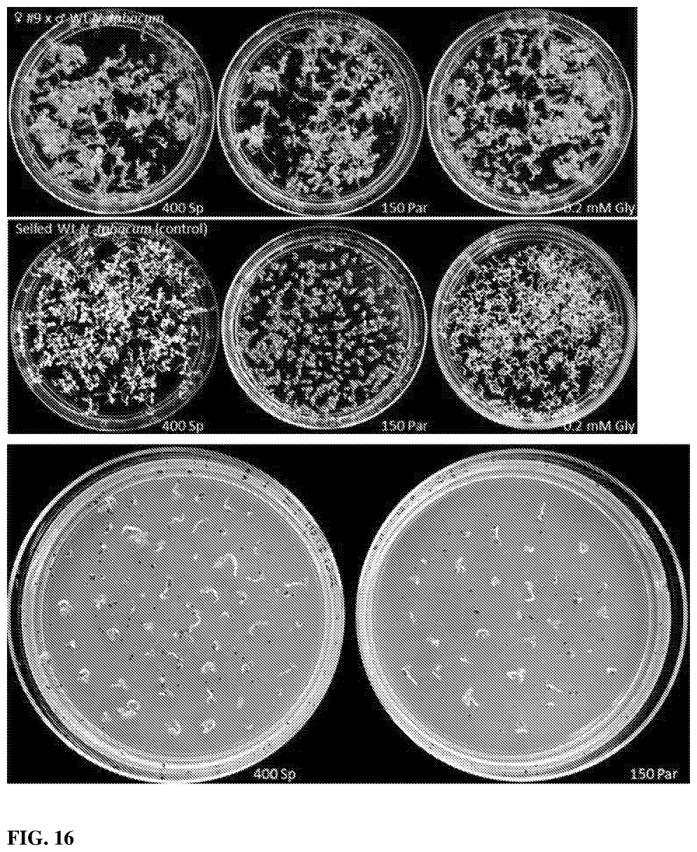

Genetic inheritance of different resistance traits from both parents were analyzed in progeny of line #9. Since the stamens of line #9 were non-functional this line was pollinated with wild type N. tabacum var. Samsun. Seeds from this cross were collected and tested on different selection media. As expected, all germinated seeds from cross ♀ #9 ♂ Wt N. tabacum var. Samsun produced green seedlings on medium with 400 mg/l Spectinomycin, 150 mg/l Paromomycin, and 0.2 mM glyphosate (FIG. 16). This demonstrates the presence in cell line #9 of traits (spectinomycin, paromomycin, and glyphosate resistance) from both parents used in this experiment and confirms that the product line #9 is indeed allopolyploid and has a combined genome from both parents.

### Example 3: Nuclear Gene Transfer by Cell Wounding and Transfer in Corn

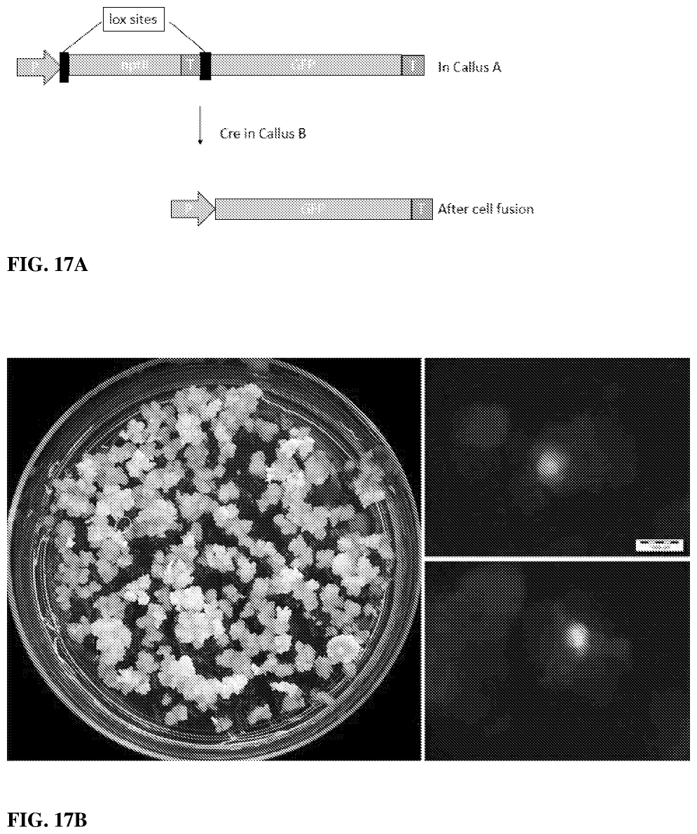

Transgenic corn line A was created having a recombinant DNA construct in its nuclear genome including, in the 5′ to 3′ direction, an enhanced CaMV 35S promoter with an HSP70 intron in the 5′ untranslated region, a nptII selectable marker gene flanked by lox sites, followed by a green fluorescent protein (GFP) gene (see, e.g., Zhang et al., Theor. Appl. Gen. 107(7): 1157-1168 (2003)). GFP is not functionally expressed due to the intervening nptII gene between the 35S promoter and the GFP coding sequence. However, in the presence of Cre recombinase enzyme, the nptII gene is excised due to the flanking lox sites, which results in high levels of GFP expression that can be visualized in most tissues by bringing the 35S promoter and the GFP coding sequence together. Embryogenic callus cells were generated from immature embryos of transgenic corn line A using methods known in the art (see, e.g., Sidorov and Duncan, Methods in Molecular Biology, Vol. 526, Transgenic Maize Methods and Protocols, Humana Press (2009)). Transgenic corn line B was established with a Cre transgene present in its nuclear genome. Embryogenic callus cells were also generated from a 7-day old seedling of transgenic line B as described before (see, e.g., Sidorov et al., Plant Cell Rep. 25: 320-328 (2006)). Thus, combining the nptII-GFP and Cre-expressing constructs in the sample plant cell will cause the nptII gene to become excised with detectable GFP expression (see FIG. 17A). According to present embodiments, these constructs present in different cells may be combined by the methods described herein.

To demonstrate exchange of nuclear genetic material between corn cells, about 1.5 g of callus cells from transgenic corn lines A and B were chopped into fine pieces, packed into clumps, and placed together on MSW57 medium supplemented with 0.5 mg/l 2,4-D and 2.2 mg/l picloram (see, e.g., Sidorov and Duncan, 2009, supra) in darkness at 28° C. As a control, the same amount of calli from transgenic corn lines A and B were mixed without wounding. The callus A and B mixtures were grown for about 2 months with regular sub-culture every 2 weeks. As shown in FIG. 17B, three independent GFP-positive colonies of cells were identified in plates from mixed cultures that were subjected to wounding, indicating exchange of material in some instances between cells from callus A and B to bring the Cre gene or expression product into a recipient cell having the nptII-GFP construct as a result of cell combination or transfer to cause excision of the nptII gene and expression of GFP coding sequence. No GFP-positive colonies were found in control plates.

While the present invention has been disclosed with reference to certain embodiments, it will be apparent that modifications and variations are possible without departing from the spirit and scope of the present invention as described herein and as provided by the appended claims. Furthermore, it should be appreciated that all examples in the present disclosure, while illustrating embodiments of the invention, are provided as non-limiting examples and are, therefore, not to be taken as limiting the various aspects so illustrated. The present invention is intended to have the full scope defined by the present disclosure, the language of the following claims, and any equivalents thereof. Accordingly, the examples, drawings and detailed description are to be regarded as illustrative and not as restrictive.

What is claimed is:

1. A method for transfer of genetic material comprising:
a) obtaining a first plant cell culture and a second plant cell culture;
b) mixing the first and second plant cell cultures to obtain a mixed cell culture; and
c) wounding the cells of the mixed cell culture to produce at least one combined cell into which transfer of a genetic material has occurred following said mixing.

2. The method of claim 1, further comprising
d) screening or selecting for the at least one combined cell, or a progeny cell thereof, or a plant developed or regenerated from the at least one combined cell, or a progeny cell thereof, based on a selectable or screenable marker.

3. The method of claim 1, wherein one or more cells of the first plant cell culture comprise a transgene, native allele, edit or mutation of interest that is not present in the cells of the second plant cell culture.

4. The method of claim 2, wherein the at least one combined cell, or a progeny cell thereof, comprises the transgene, native allele, edit or mutation of interest present in the one or more cells of the first plant cell culture.

5. The method of claim 1, wherein the first and second plant cell cultures are callus cultures or cell suspension cultures.

6. The method of claim 1, wherein at least one of the first and second plant cell cultures comprises cells having a plastid genome-encoded marker gene, and/or wherein at least one of the first and second plant cell cultures comprise cells having a nuclear genome-encoded marker gene.

7. The method of claim 6, wherein the first and second plant cell cultures each comprise cells having a plastid genome-encoded marker gene.

8. The method of claim 6, wherein the first and second plant cell cultures each comprise cells having a nuclear genome-encoded marker gene.

9. The method of claim 6, wherein the first plant cell culture comprises cells having a plastid genome-encoded marker gene, and the second plant cell culture comprises cells having a nuclear genome-encoded marker gene.

10. The method of claim 6, further comprising
d) screening or selecting for the at least one combined cell of the mixed cell culture, or at least one progeny cell thereof, or a plant developed or regenerated from the at least one combined cell, or a progeny cell thereof, based on the presence of the plastid genome-encoded marker gene, during and/or after step (c).

11. The method of claim 6, further comprising
d) screening or selecting for the at least one combined cell of the mixed cell culture, or at least one progeny cell thereof, or a plant developed or regenerated from the at least one combined cell, or a progeny cell thereof, based on the presence of the nuclear genome-encoded marker gene, during and/or after step (c).

12. The method of claim 1, further comprising
d) regenerating a plant from the mixed cell culture and/or the at least one combined cell, or at least one progeny cell thereof.

13. The method of claim 2, further comprising
e) regenerating a plant from the mixed cell culture and/or the at least one combined cell, or at least one progeny cell thereof.

14. The method of claim 6, wherein cells of the first and/or second plant cell cultures are dicot plant cells.

15. The method of claim 14, wherein the dicot plant cells are selected from the group consisting of tobacco, tomato, soybean, canola, and cotton cells.

16. The method of claim 6, wherein cells of the first and/or second plant cell cultures are monocot plant cells.

17. The method of claim 16, wherein the monocot plant cells are selected from the group consisting of corn, rice, wheat, barley, and sorghum cells.

18. The method of claim 6, wherein the plastid genome-encoded marker gene is a selectable marker gene.

19. The method of claim 18, wherein the selectable marker gene is selected from the group consisting of: aadA, rrnS, rrnL, nptII, aphA-6, psbA, bar, HPPD, ASA2, and AHAS.

20. The method of claim 6, wherein the plastid genome-encoded marker gene is a screenable marker gene.

21. The method of claim 20, wherein the screenable marker gene is gfp or gus.

22. The method of claim 6, wherein the nuclear genome-encoded marker gene is a selectable marker gene.

23. The method of claim 22, wherein the selectable marker gene is selected from the group consisting of: nptII, EPSPS, bar, hpt, dmo, and GAT.

24. The method of claim 6, wherein the nuclear genome-encoded marker gene is a screenable marker gene.

25. The method of claim 24, wherein the screenable marker gene is selected from the group consisting of: uidA (gus) and gfp.

26. The method of claim 1, wherein a first cell of the first plant cell culture is a donor cell and a second cell of the second plant cell culture is a recipient cell.

27. The method of claim 1, wherein cells of the first and second plant cell cultures have the same ploidy level.

28. The method of claim 1, wherein the combined cell and cells of one or both of the first and/or second plant cell cultures have the same ploidy level.

29. The method of claim 6, wherein cells of at least one of the first and second plant cell cultures comprise a plastid genome-encoded marker gene, and wherein cells of at least one of the first and second plant cell cultures comprise a nuclear genome-encoded marker gene.

30. The method of claim 5, wherein the cells of the mixed cell culture, or progeny cells thereof, are screened or selected for the presence of a marker gene encoded by a nuclear genome-encoded gene, during and/or after step (c.

31. The method of claim 1, wherein the cells of the mixed cell culture, the first plant cell culture and/or the second plant cell culture, or progeny cells thereof, are homoplastomic for a plastid-encoded gene.

32. The method of claim 1, wherein the cells of the mixed cell culture, the first plant cell culture and/or the second plant cell culture, or progeny cells thereof, are heteroplastomic for a plastid-encoded gene.

33. A combined plant cell produced by the method of claim 1.

34. The combined plant cell of claim 33, wherein the plant cell is a dicot plant cell.

35. The dicot plant cell of claim 34, selected from the group consisting of: a tobacco, a tomato, a soybean, a canola, and a cotton plant cell.

36. The combined plant cell of claim 33, wherein the combined plant cell is a monocot plant cell.

37. The monocot plant cell of claim 36, selected from the group consisting of: a corn, a rice, a wheat, and a sorghum plant cell.

38. A plant regenerated from the combined plant cell produced by the method of claim 1, or a progeny cell thereof.

39. The regenerated plant of claim 38, wherein the plant is a dicot plant.

40. A seed, progeny plant, or progeny seed of the plant of claim 39.

41. The dicot plant of claim 39, selected from the group consisting of: a tobacco, a tomato, a soybean, a canola, and a cotton plant.

42. The regenerated plant of claim 38, wherein the plant is a monocot plant.

43. A seed, progeny plant or progeny seed of the plant of claim 42.

44. The monocot plant of claim 42, selected from the group consisting of: a corn, a rice, a wheat, a barley, and a sorghum plant.

45. A wounded mixed cell culture produced by the method of steps (a)-(c) of claim 1.

46. The method of claim 1, wherein the genetic transfer comprises plastid or organellar gene transfer.

47. The method of claim 1, wherein the genetic transfer comprises nuclear gene transfer.

48. A method for transfer of genetic material comprising:
a) obtaining a first plant cell culture and a second plant cell culture;
b) wounding the cells of one or both of the first and second plant cell cultures; and
c) mixing the first and second plant cell cultures to obtain a mixed cell culture to produce at least one combined cell into which transfer of a genetic material has occurred.

49. The method of claim 48, further comprising
d) screening or selecting for the at least one combined cell, or a progeny cell thereof, or a plant developed or regenerated from the at least one combined cell, or a progeny cell thereof, based on a selectable or screenable marker.

50. The method of claim 48, wherein the first and second plant cell cultures are callus cultures or cell suspension cultures.

51. The method of claim 48, wherein at least one of the first and second plant cell cultures comprises cells having a plastid genome-encoded marker gene, and/or wherein at least one of the first and second plant cell cultures comprise cells having a nuclear genome-encoded marker gene.

52. The method of claim 51, wherein the first and second plant cell cultures each comprise cells having a plastid genome-encoded marker gene.

53. The method of claim 51, wherein the first and second plant cell cultures each comprise cells having a nuclear genome-encoded marker gene.

54. The method of claim 51, wherein the first plant cell culture comprises cells having a plastid genome-encoded marker gene, and the second plant cell culture comprises cells having a nuclear genome-encoded marker gene.

55. The method of claim 51, further comprising
d) screening or selecting for the at least one combined cell of the mixed cell culture, or at least one progeny cell thereof, or a plant developed or regenerated from the at least one combined cell, or a progeny cell thereof, based on the presence of the plastid genome-encoded marker gene, during and/or after step (c).

56. The method of claim 51, further comprising
d) screening or selecting for the at least one combined cell of the mixed cell culture, or at least one progeny cell thereof, or a plant developed or regenerated from the at least one combined cell, or a progeny cell thereof, based on the presence of the nuclear genome-encoded marker gene, during and/or after step (c).

57. The method of claim 55 or 56, further comprising
e) regenerating a plant from the mixed cell culture and/or the at least one combined cell, or at least one progeny cell thereof.

58. The method of claim 51, wherein cells of the first and/or second plant cell cultures are dicot plant cells.

59. The method of claim 51, wherein cells of the first and/or second plant cell cultures are monocot plant cells.

60. The method of claim 51, wherein the plastid genome-encoded marker gene is a selectable or screenable marker gene.

61. The method of claim 51, wherein the nuclear genome-encoded marker gene is a selectable or screenable marker gene.

62. The method of claim 48, wherein a first cell of the first plant cell culture is a donor cell and a second cell of the second plant cell culture is a recipient cell.

63. The method of claim 48, wherein cells of the first and second plant cell cultures have the same ploidy level.

64. The method of claim 48, wherein the combined cell and cells of one or both of the first and/or second plant cell cultures have the same ploidy level.

65. The method of claim 51, wherein cells of at least one of the first and second plant cell cultures comprise a plastid genome-encoded marker gene, and wherein cells of at least one of the first and second plant cell cultures comprise a nuclear genome-encoded marker gene.

66. The method of claim 49, wherein the cells of the mixed cell culture, or progeny cells thereof, are screened or selected for the presence of a marker gene encoded by a nuclear genome-encoded gene, during and/or after step (c) or (d).

67. A combined plant cell produced by the method of claim 48.

68. The combined plant cell of claim 67, wherein the plant cell is a dicot plant cell.

69. The dicot plant cell of claim 68, selected from the group consisting of: a tobacco, a tomato, a soybean, a canola, and a cotton plant cell.

70. The combined plant cell of claim 67, wherein the combined plant cell is a monocot plant cell.

71. The monocot plant cell of claim 70, selected from the group consisting of: a corn, a rice, a wheat, and a sorghum plant cell.

72. A plant regenerated from the combined plant cell produced by the method of claim 48, or a progeny cell thereof.

73. A seed, progeny plant, or progeny seed of the plant of claim 72.

74. A wounded mixed cell culture produced by the method of steps (a)-(c) of claim 48.

75. The method of claim 48, wherein the genetic transfer comprises plastid or organellar gene transfer.

76. The method of claim 48, wherein the genetic transfer comprises nuclear gene transfer.

77. A method for editing a plant cell comprising:
a) obtaining a first plant cell culture and a second plant cell culture, wherein one or more cells of the first plant cell culture comprise a recombinant DNA transgene comprising a sequence encoding a site-specific nuclease operably linked to a first promoter;
b) mixing the first and second plant cell cultures to obtain a mixed cell culture; and
c) wounding the cells of the mixed cell culture to produce at least one edited product cell having an edit or mutation introduced in its genome by the site-specific nuclease.

78. A method for editing a plant cell comprising:
a) obtaining a first plant cell culture and a second plant cell culture, wherein one or more cells of the first plant cell culture comprise a recombinant DNA transgene comprising a sequence encoding a site-specific nuclease operably linked to a first promoter;
b) wounding the cells of one or both of the first and second plant cell cultures; and
c) mixing the first and second plant cell cultures to obtain a mixed cell culture to produce at least one edited product cell having an edit or mutation introduced in its genome by the site-specific nuclease.

79. The method of claim 77 or 78, further comprising:
d) screening or selecting for the at least one edited product cell, or a progeny cell thereof, or a plant developed or regenerated from the at least one edited product cell, or a progeny cell thereof, having the edit or mutation.

80. The method of claim 79, wherein the plant developed or regenerated from the at least one edited product cell, or a progeny cell thereof, is screened or selected based on a trait or phenotype produced by the edit or mutation and present in the developed or regenerated plant, or a progeny plant, plant part or seed thereof.

81. The method of claim 79, wherein the at least one edited product cell, or a progeny cell thereof, or the plant developed or regenerated from the at least one edited product cell, or a progeny cell thereof, are screened or selected based on a molecular assay.

82. The method of claim 77 or 78, wherein the first and second plant cell cultures are callus cultures or cell suspension cultures.

83. The method of claim 77 or 78, further comprising
d) regenerating a plant from the mixed cell culture and/or the at least one edited product cell, or at least one progeny cell thereof.

84. The method of claim 79, further comprising
e) regenerating a plant from the mixed cell culture and/or the at least one edited product cell, or at least one progeny cell thereof.

85. The method of claim 77 or 78, wherein cells of the first and/or second plant cell cultures are dicot plant cells.

86. The method of claim 85, wherein the dicot plant cells are selected from the group consisting of tobacco, tomato, soybean, canola, and cotton cells.

87. The method of claim 77 or 78, wherein cells of the first and/or second plant cell cultures are monocot plant cells.

88. The method of claim 87, wherein the monocot plant cells are selected from the group consisting of corn, rice, wheat, barley, and sorghum cells.

89. The method of claim 77 or 78, wherein a first cell of the first plant cell culture is a donor cell and a second cell of the second plant cell culture is a recipient cell.

90. The method of claim 77 or 78, wherein the first promoter operably linked to the sequence encoding a site-specific nuclease is a constitutive promoter, a tissue-specific or tissue-preferred promoter, a developmental stage promoter, or an inducible promoter.

91. The method of claim 77 or 78, wherein the site-specific nuclease is a zinc-finger nuclease (ZFN), a meganuclease, an RNA-guided endonuclease, a TALE-endonuclease (TALEN), a recombinase, or a transposase.

92. The method of claim 91, wherein the site-specific nuclease is an RNA-guided nuclease.

93. The method of claim 77 or 78, wherein the one or more cells of the first plant cell culture further comprise a first recombinant DNA construct comprising a first transcribable DNA sequence encoding a guide RNA molecule operably linked to a promoter.

94. The method of claim 93, wherein the promoter operably linked to the first transcribable DNA sequence is a constitutive promoter, a tissue-specific or tissue-preferred promoter, a developmental stage promoter, or an inducible promoter.

95. The method of claim 77 or 78, wherein the one or more cells of the first plant cell culture further comprise a second recombinant DNA construct comprising a second transcribable DNA sequence encoding a donor template molecule operably linked to a promoter.

96. The method of claim 95, wherein the donor template molecule comprises a transgene comprising a coding sequence or transcribable DNA sequence operably linked to a plant-expressible promoter.

97. The method of claim 95, wherein the promoter operably linked to the second transcribable DNA sequence is a constitutive promoter, a tissue-specific or tissue-preferred promoter, a developmental stage promoter, or an inducible promoter.

98. The method of claim 77 or 78, wherein one or more cells of the second plant cell culture comprise a recombinant DNA construct comprising a first transcribable DNA sequence encoding a guide RNA molecule operably linked to a promoter.

99. The method of claim 77 or 78, wherein one or more cells of the second plant cell culture comprise a recombinant DNA construct comprising a second transcribable DNA sequence encoding a donor template molecule operably linked to a promoter.

100. The method of claim 99, wherein the donor template molecule comprises a transgene comprising a coding sequence or transcribable DNA sequence operably linked to a plant-expressible promoter.

101. An edited product cell produced by the method of claim 77 or 78.

102. The edited product cell of claim 101, wherein the plant cell is a dicot plant cell.

103. The edited product cell of claim 101, wherein the plant cell is a monocot plant cell.

104. A plant regenerated or developed from the edited product cell produced by the method of claim 77 or 78, or a progeny cell thereof.

105. The regenerated plant of claim 104, wherein the plant is a dicot or monocot plant.

106. A seed, progeny plant, or progeny seed of the plant of claim 105.

107. A wounded mixed cell culture produced by the method of steps (a)-(c) of claim 77 or 78.
